(19) United States
(12) Patent Application Publication
(10) Pub. No.: US 2016/0060711 A1
(43) Pub. Date: Mar. 3, 2016

(54) CruP Protects Against ROS in Oxygenic Photosynthetic Organisms

(71) Applicant: Research Foundation of The City University of New York, New York, NY (US)

(72) Inventors: Eleanore T. Wurtzel, Great Neck, NY (US); Louis Bradbury, New York, NY (US)

(21) Appl. No.: 14/403,208

(22) PCT Filed: May 21, 2013
(86) PCT No.: PCT/US2013/042010

§ 371 Date: Nov. 24, 2014

(51) Int. Cl. C12Q 1/68; C12N 15/82

(52) U.S. Cl. CPC C12Q 1/6895; C12N 15/8273; C12N 15/8271; C12Q 2600/13; C12Q 2600/158

(57) ABSTRACT

The herein invention provides methods for selecting a plant that is cold and anoxia tolerant by detecting the expression of CruP in said plant, selecting a plant that overexpresses CruP as being cold and anoxia tolerant. Also provided are methods for providing cold tolerance, anoxia tolerance and protection against reactive oxygen species in a plant by introducing a gene that encodes CruP with higher than wild type expression in said plant.

This application is a National Stage application of International Application No. PCT/US2013/042010, filed May 21, 2013, which claims benefit to Provisional Patent Application No. 61/649,961, filed May 22, 2012, which are incorporated by reference herein in its entirety.

This invention is supported by grants from the National Institutes of Health (GM081160) and NIH-NHLBI grant 5SC1HL096016.

### BACKGROUND OF THE INVENTION

Carotenoids are C₄₀ compounds found in a wide variety of organisms where they play important roles in photo-protection and light harvesting. In photosynthetic organisms, carotenoids with cyclic end groups are essential for light harvesting. Lycopene, a linear carotenoid, is the major branch point for the formation of different cyclic carotenoids such as α- or β-carotene. The cyclization of the ends of lycopene is performed by a class of enzymes known as lycopene cyclases. In plants, the enzymatic products of the CrtL type lycopene cyclases, lycopene ε-cyclase (LCYE) and lycopene β-cyclase (LCYB) are α- and β-carotene. These carotenoids can be hydroxylated to generate lutein and zeaxanthin, respectively. Lutein functions in the assembly of the photosystems and plays a role together with zeaxanthin in light harvesting within the antenna of photosystems I & II (PSI & PSII). β-carotene found in the reaction center of PSII has a protective role, quenching singlet oxygen generated during the water splitting process of photosynthesis.

Various structural types of lycopene cyclases have been identified in carotenogenic organisms, such as the CrtL type found in plants, algae and some cyanobacteria, the CrtY type in flavobacteria, and a heterodimeric type found in some gram-positive bacteria and fungi. Recently a fourth family of lycopene cyclases was identified in Green Sulfur Bacteria (GSB) and some cyanobacteria. This new family of lycopene cyclases is composed of two classes of enzymes, one known as CruA (found in all GSB and cyanobacteria that lack CrtL) and a paralog known as CruP, found in cyanobacteria, along with CruA or CrtL, and in higher plants along with CrtL enzymes (LCYB and LCYE).

In Escherichia coli complementation assays, CruA from the GSB Chlorobium tepidum (_{Ct}CruA) was shown to convert lycopene into γ-carotene plus small amounts of β-carotene. The cyanobacterial Synechococcus sp. PCC 7002 CruP (_{Syn}CruP) was also shown to convert lycopene into γ-carotene but had lower activity than _{Ct}CruA. In general, carotenoid biosynthesis in plants and cyanobacteria is performed by a similar suite of enzymes. Although plants already contain two CrtL type lycopene cyclases that form □- and □-rings, it has been suggested that CruP in plants might be a lycopene cyclase specifically responsible for catalyzing formation of β rings of α-carotene. However, the pigment profile of both the Synechococcus sp. PCC 7002 and the Synechocystis sp. PCC 6803 CruP knockouts were phenotypically identical to wild type, which is unexpected if CruP is indeed a lycopene cyclase.

In photosynthetic organisms, carotenoids serve essential roles in photosynthesis and photoprotection. A previous report designated CruP as a secondary lycopene cyclase involved in carotenoid biosynthesis. However, it has been discovered by Applicants that CruP knockout or CruP over-expression plants do not exhibit correspondingly reduced or increased production of cyclized carotenoids, which would be expected if CruP was a lycopene cyclase. Instead, it has been surprisingly discovered that CruP aids in preventing accumulation of reactive oxygen species (ROS) thereby reducing accumulation of β-carotene-5,6-epoxide, a ROS-catalyzed autoxidation product and inhibiting accumulation of anthocyanins, known chemical indicators of ROS.

As a result of Applicants discovery, plants with a non-functional CruP accumulate substantially higher levels of ROS and β-carotene-5,6-epoxide in green tissues. Plants over-expressing CruP show reduced levels of ROS, β-carotene-5,6-epoxide and anthocyanins. The observed up-regulation of CruP transcripts under photoinhibitory and lipid peroxidation-inducing conditions, such as high light stress, cold stress, anoxia and low levels of CO₂, fits with a role for CruP in mitigating the effects of ROS. Phylogenetic distribution of CruP in prokaryotes showed that the gene is only present in cyanobacteria that live in habitats characterized by large variation in temperature and inorganic carbon availability. Therefore, CruP represents a new target for developing resilient plants and algae needed to supply food and biofuels in the face of global climate change.

### SUMMARY OF THE INVENTION

The present invention provides a method for selecting a plant that is cold and anoxia tolerant. The method comprises detecting the expression of CruP in a plant, and selecting a plant that overexpresses CruP as being cold and anoxia tolerant. In one embodiment, the plant is algae.

A further aspect of the invention is a method for providing cold tolerance to a plant. The method comprises introducing a gene that encodes CruP with higher than wild-type expression into a plant. One variety of plant is algae.

In another embodiment, a method for providing anoxia tolerance to a plant is presented. The method comprises introducing a gene that encodes CruP with higher than wild type expression into a plant. The plant can be algae.

Finally, a method for providing protection against reactive oxygen species in a plant is presented. The method comprises introducing a gene that encodes CruP with higher than wild type expression in a plant. In one embodiment, the plant is algae.

### BRIEF DESCRIPTION OF THE FIGURES

FIG. 1. HPLC analysis of carotenoids extracted from E. coli BL21 cells containing pAC-CRT-EIB and A) Empty pET16b vector, B) pET-_{Syn}CruP and C) p16-CPL1 (_{Cp}CruA). Absorbance spectrum of D) peak 1 (lycopene) and E) peak 2 (γ-carotene) is also shown.

FIG. 2. Localization of _{Zm}CruP in chloroplasts. A) Signal detected from SDS-PAGE gel of radiolabeled _{Zm}CruP before (P) and after (I) import into chloroplasts. T—Thermolysin treated fraction. S— Soluble fraction. M—Membrane fraction. A—Alkaline treated membrane fraction. B) Transient expression of _{Zm}CruP:GFP under a 35S promoter in bean cotyledon protoplasts showing GFP fluorescence, chlorophyll fluorescence, a merged image showing both GFP and chlorophyll fluorescence and a bright field image showing intact protoplast.

FIG. 3. Two-week-old plants grown on MS media. A) Columbia wild type, B) _{At}CruP knockout.

FIG. 4. HPLC analysis of carotenoids extracted from Arabidopsis plants. A) Columbia wild type, B) _{At}CruP knockout plants, C) Absorbance spectra of the unique peak (*) identified in the _{At}CruP knockout plants.

FIG. 5. HPLC analysis of carotenoids extracted from a segregating population of Arabidopsis plants. A) Typical homozygous wild type plant, B) Typical heterozygous _{At}CruP knockout plant, C) Typical homozygous _{At}CruP knockout plant, D) Typical heterozygous _{At}CruP knockout/Hemizygous 35S:_{Zm}CruP plant, E) Absorbance spectra of the unique peak (*) identified in the _{At}CruP knockout plants, F) Absorbance spectra of the unknown chlorophyll-like peak (Chl), G) Absorbance spectra of β-carotene (β). Unknown cis-carotenoids were also observed to elute between Chl and β.

FIG. 6. Comparison of HPLC elution profiles of A) pigments extracted from a homozygous _{At}CruP knockout plant to B) synthesized β-carotene-5,6-epoxide standard. C) Absorbance spectra of the unknown peak from the _{At}CruP knockout plants. D) Absorbance spectra of the synthesized β-carotene-5,6-epoxide standard. E) Chemical structure of β-carotene-5,6-epoxide.

FIG. 7. Levels of β-carotene and β-carotene-5,6-epoxide displayed as a ratio of total chlorophyll from leaves of Arabidopsis plants (Columbia wild type and _{At}CruP knockout) grown under different temperatures.

FIG. 8. ROS levels in cotyledons shown as a percent area of cotyledons stained by nitrotetrazolium blue. Lines used are Columbia wild type, CruP knockout and 35S:ZmCruP lines p1, pX1, pX2 and p5.

FIG. 9. Arabidopsis plants shifted to 4° C. with continuous light for 2 months showing production of (or lack of) anthocyanin in response to this stress. A) Columbia wild type, B) _{At}CruP knockout and C-E) 35S:_{Zm}CruP Arabidopsis plants pX1, pX2 and p5 respectively.

FIG. 10. A phylogenetic tree based on 16S rRNA sequences of completely sequenced cyanobacteria using A. thaliana mitochondrial 16S rRNA as an outlier. Bootstrap values shown are based on 1000 replicates. Columns to the right of the tree show the GenBank protein IDs of CruP, CruA, LCYB, CcmN and CsoS2 if it is present in that organism.

FIG. 11. CruP genes (red arrow) of select cyanobacteria showing clustering with genes, the products of which are involved in carbon acquisition (blue arrows), PSII reaction center (green arrows) and PSII reaction center repair (purple arrows).

FIG. 12. Carotenoid gene transcripts up-regulated more than 1.5 fold by A) chilling stress and B) anoxia. Data obtained from published microarray data available via Genevestigator.

FIG. 13. Phylogenetic distribution of CruA, CrtY and CrtL-type cyclases and CruP proteins.

### DETAILED DESCRIPTION OF THE INVENTION

As a result of the herein invention, it has been shown that the absence of CruP is associated with increased reactive oxygen species (ROS) and increased β-carotene-5,6-epoxide, while overexpression of CruP is associated with reduced ROS, reduced β-carotene-5,6-epoxide and a significantly reduced anthocyanin response under cold stress. The above results suggest that the function of CruP is to reduce oxidative damage caused by singlet oxygen.

Thus, CruP represents a new target for developing resilient plants and algae needed to supply food and biofuels in the face of global climate change. The up-regulation of CruP during cold and anoxic conditions, such as flooding, suggests also that CruP will be an important locus to consider in screening for cold and submergence (anoxia) tolerance in plants.

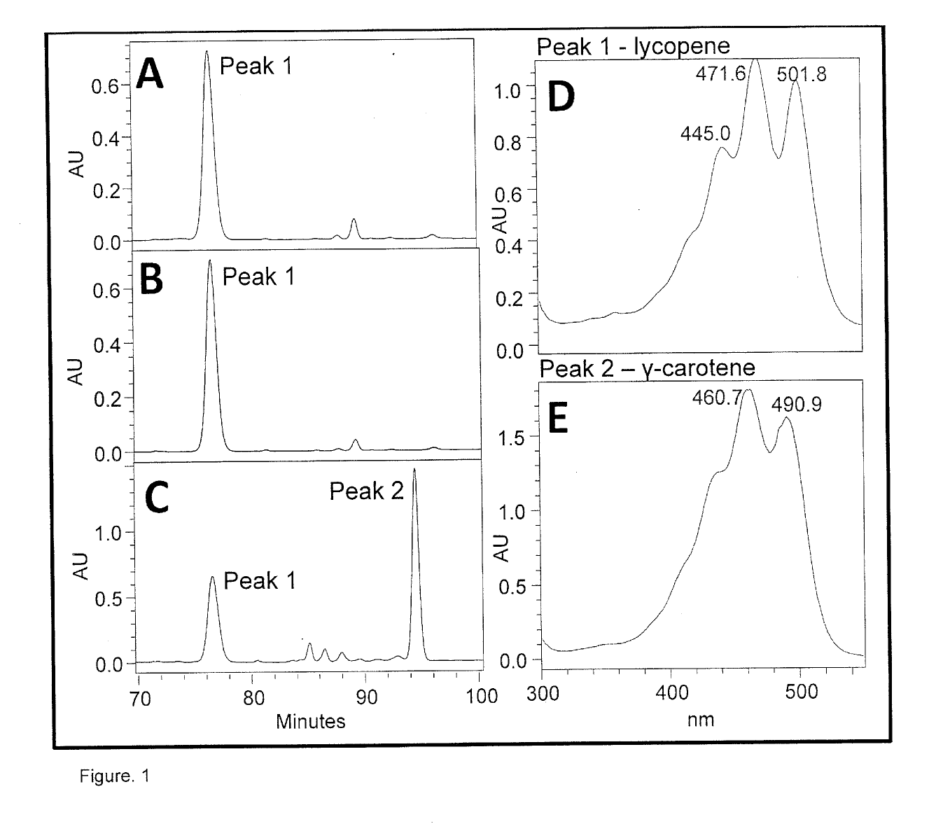

Published reports have indicated that _{Syn}CruP is a lycopene cyclase. To confirm this, both _{Syn}CruP and cruA from Chlorobium phaeobacteroides (_{Cp}cruA) in E. coli BL21 containing pAC-CRT-EIB which confers lycopene accumulation, were expressed. In this system, a functional lycopene β cyclase converts lycopene into γ- or β-carotene. However, expression of pET16-_{Syn}CruP in this lycopene accumulating strain of E. coli revealed that, despite high production of CruP protein, there was no change in the pigments produced in comparison to a strain containing pAC-CRT-EIB and the empty pET16b vector (FIGS. 1 A&B). In contrast, expression of p16-CPL1 containing _{Cp}CruA led to conversion of all of the lycopene into γ-carotene (FIG. 1 C). Therefore only CruA, and not CruP, appears to have lycopene cyclase activity in E. coli.

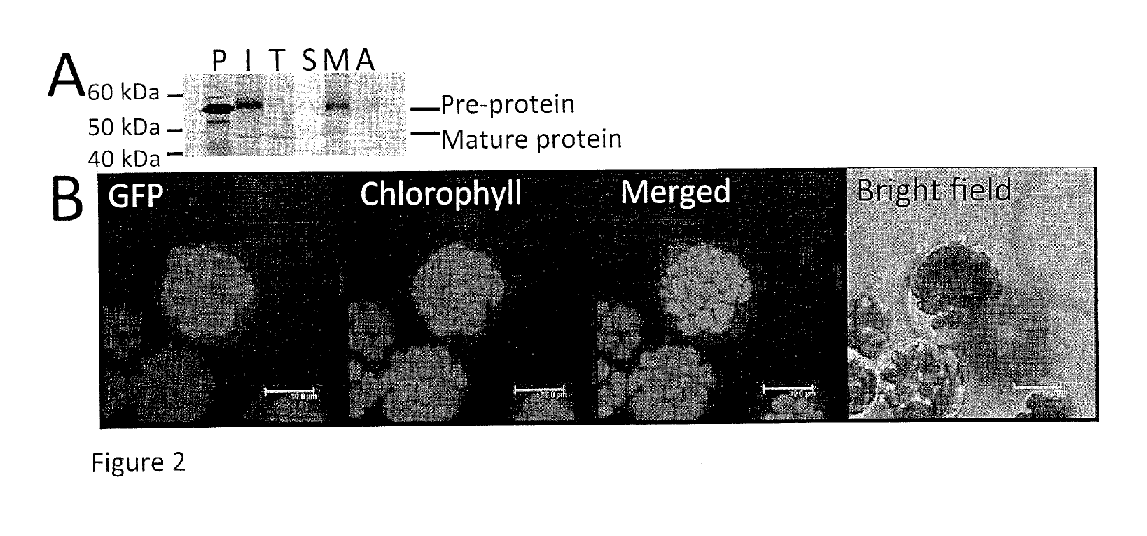

If CruP were a lycopene cyclase, it would be localized to chloroplasts, the site of carotenoid biosynthesis. Therefore, the location of Zea mays CruP (_{Zm}CruP) in chloroplasts via in vitro import experiments was tested. Incubation of _{Zm}CruP with isolated pea chloroplasts led to import and processing of the _{Zm}CruP precursor protein (large band in FIG. 2A, P) to generate a smaller mature protein (smaller band in FIG. 2A, I). After thermolysin treatment of the chloroplasts (FIG. 2A, T) only the smaller protein remained, showing that the mature protein is completely inside the chloroplast and is protected by the outer membrane. Fractionation of non-thermolysin treated chloroplasts showed _{Zm}CruP is present in the membrane fraction (FIG. 2A, M) and is absent from the soluble fraction (FIG. 2A, S). After alkaline treatment (FIG. 2A, A) the membrane fraction was devoid of _{Zm}CruP showing it is a peripherally membrane bound chloroplast protein. Chloroplast localization of _{Zm}CruP was further corroborated by transient expression in maize leaf protoplasts using a _{Zm}CruP:GFP fusion driven by a 35S promoter. The GFP fluorescence of the fusion protein was localized with chlorophyll fluorescence (FIG. 2B). Therefore the import and transient expression experiments demonstrate chloroplast localization of _{Zm}CruP. The precise suborganellar location is likely to be thylakoids as suggested by proteomic surveys conducted in Arabidopsis (The Plant Proteome Database, http://ppdb.tc.cornell.edu/dbsearch/searchsample.aspx).

To gain further insight into the function of CruP, _{At}CruP gene expression was compared to expression of other genes encoding carotenoid enzymes in Arabidopsis. The expression profile of _{At}CruP analyzed using Genevestigator®, showed that _{At}CruP is expressed highest in green tissues (e.g. pedicels, sepals, cotyledons and young leaves). _{At}CruP is up-regulated by light as seen for carotenoid-related genes. However, in contrast to most carotenoid-related genes, _{At}CruP is expressed at relatively low levels under most conditions except cold stress and dark anoxic treatment where _{At}CruP is highly expressed (FIG. 14). Most other stress stimuli data available on Genevestigator®, including drought and ABA treatment, either do not alter expression or decrease expression of _{At}CruP. Therefore, transcript levels of CruP appear to be controlled independently of carotenoid pathway genes, which would be consistent with a role of CruP distinct from carotenogenesis.

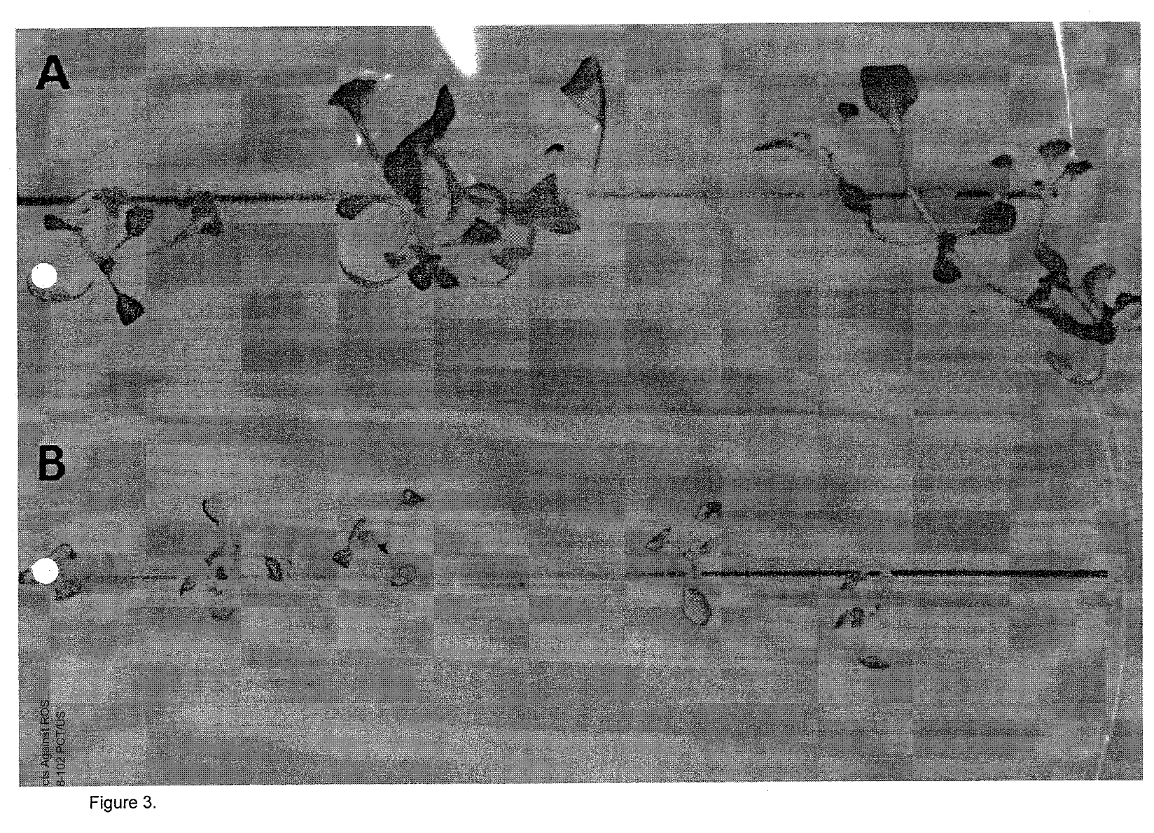

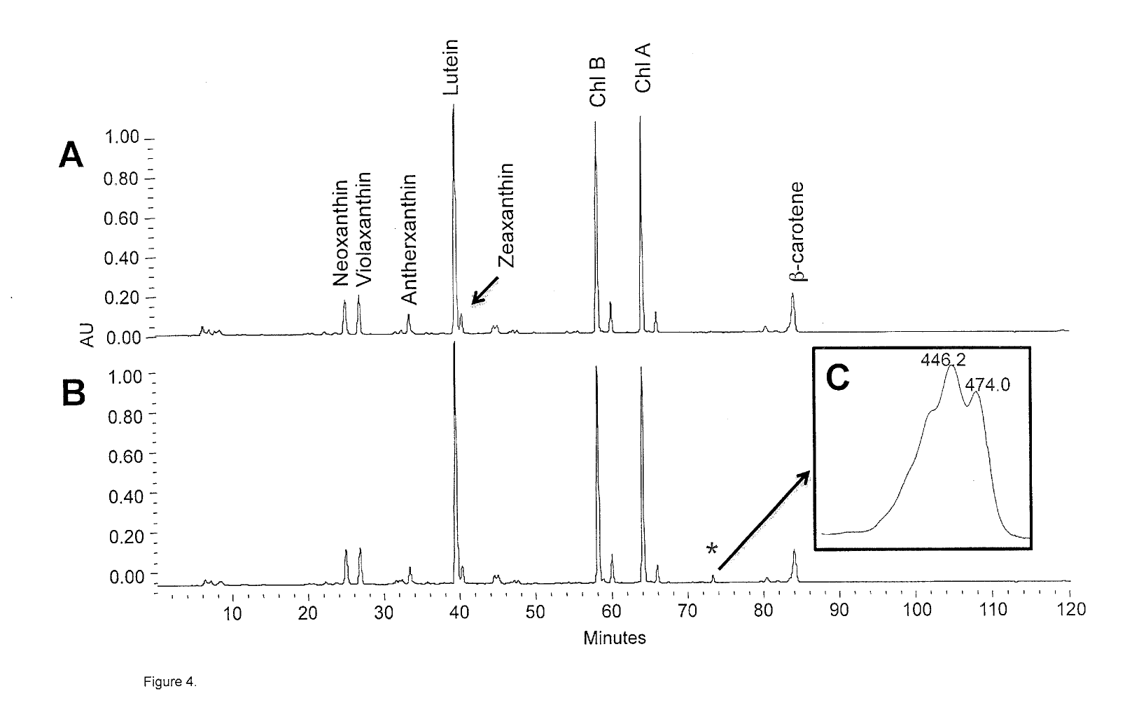

Photosynthetic organisms that lack lycopene cyclase activity exhibit accumulation of lycopene along with aberrant chloroplast ultrastructure, which appears not to be the case for CruP mutants. Previous reports of phenotypes from cru knockouts in cyanobacteria range from no phenotype to descriptions of disordered thylakoid membranes. In both cases, no change in carotenoid pigment profile was observed. To test whether plant mutants of CruP might exhibit evidence of lycopene cyclase activity, the pigment profiles of CruP knockout and over-expressing Arabidopsis plants were analyzed. RT-PCR confirmed an absence of _{At}CruP transcripts in the knockout line (SALK_{—}011725) and over-expression of _{Zm}CruP transcripts in the four 35S:_{Zm}CruP transgenic lines. The pigment profile of the knockout plants showed no change in levels of lutein (the hydroxylation product of α-carotene), suggesting CruP was not involved in the production of α-carotene, as had been previously suggested. The only consistent difference observed was that of a small peak barely noticeable in the wild type, which was found to be present at levels roughly ten times higher in the _{At}CruP knockout plants. It is also noted that the growth rate of the knockout plants was significantly slower than wild type plants (FIG. 3). At 2 weeks growth on MS medium, wild type plants had on average seven leaves while the knockout plants had on average only four leaves.

To confirm that the presence of the unknown peak was not due to another random mutation within this knockout line, a segregating population was obtained by crossing the knockout line with the Columbia wild type, followed by selfing of the progeny. Eight homozygous knockouts obtained from this cross were analyzed by HPLC and all were found to contain the unknown peak (FIG. 5 C). In contrast, this peak was virtually absent in all of the seven analyzed wild type plants generated from this cross (FIG. 5 A). Three heterozygous plants were also analyzed and showed half as much of the unknown peak as the homozygous knockout plants (FIG. 5 B). We also crossed lines to produce plants that were heterozygous for the _{At}CruP knockout and hemizygous for 35S:_{Zm}CruP, the pigment profile of these plants was examined and showed the complete absence of the unknown peak (FIG. 5 D).

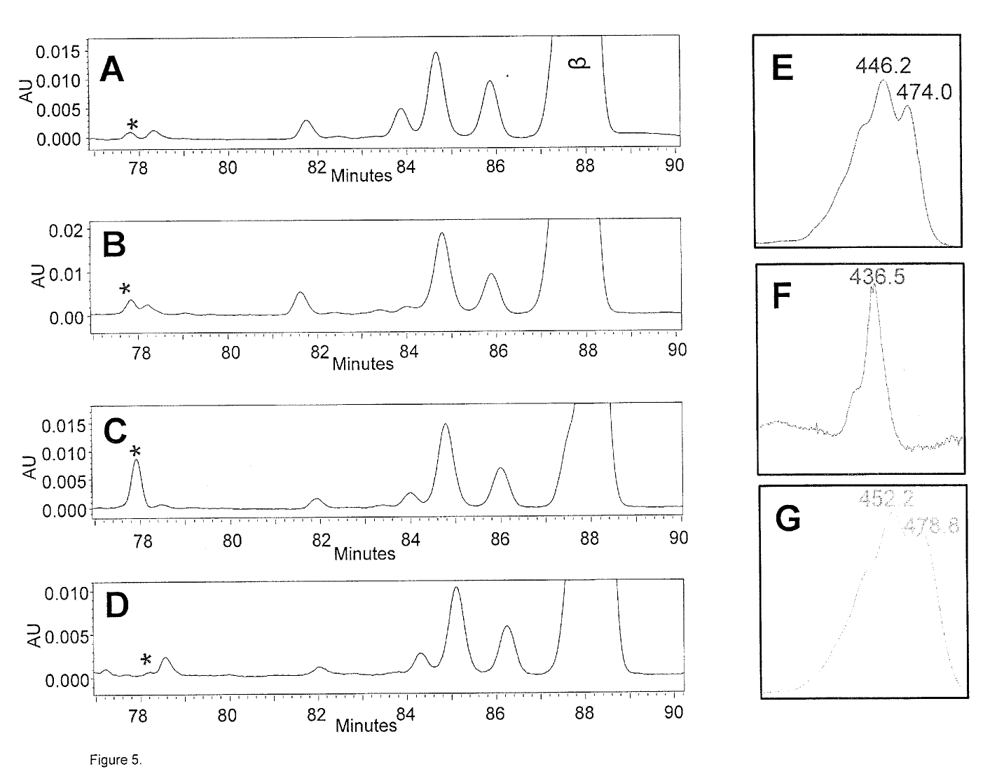

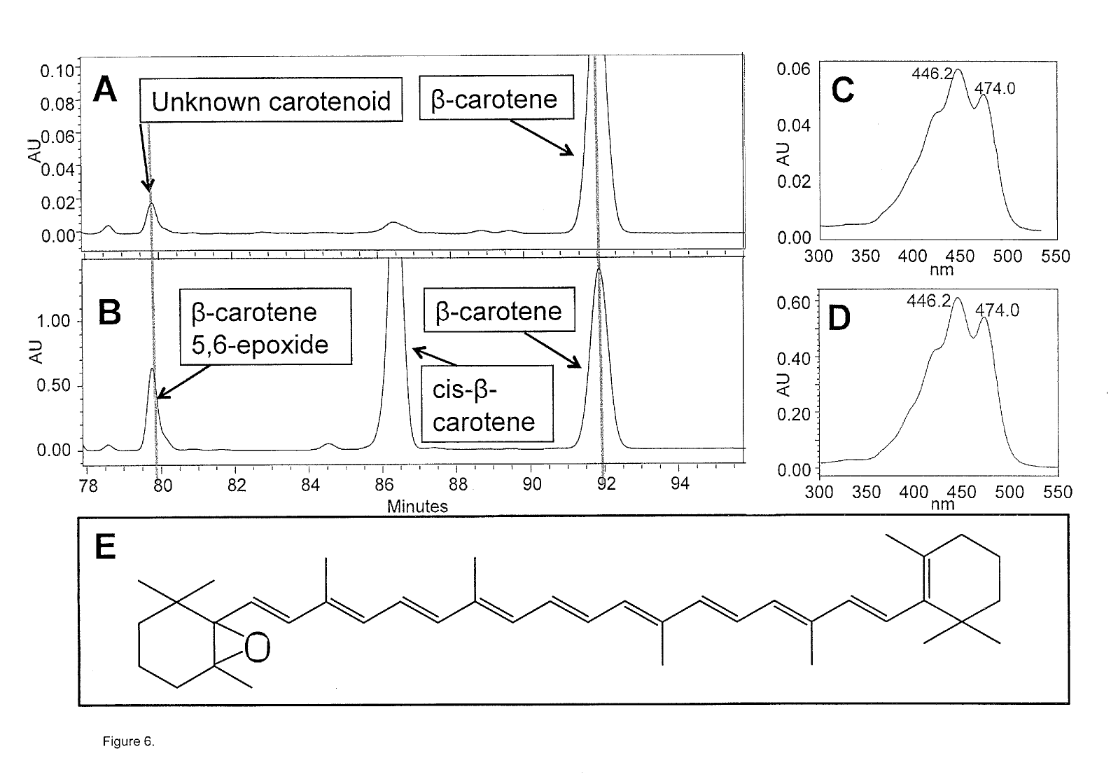

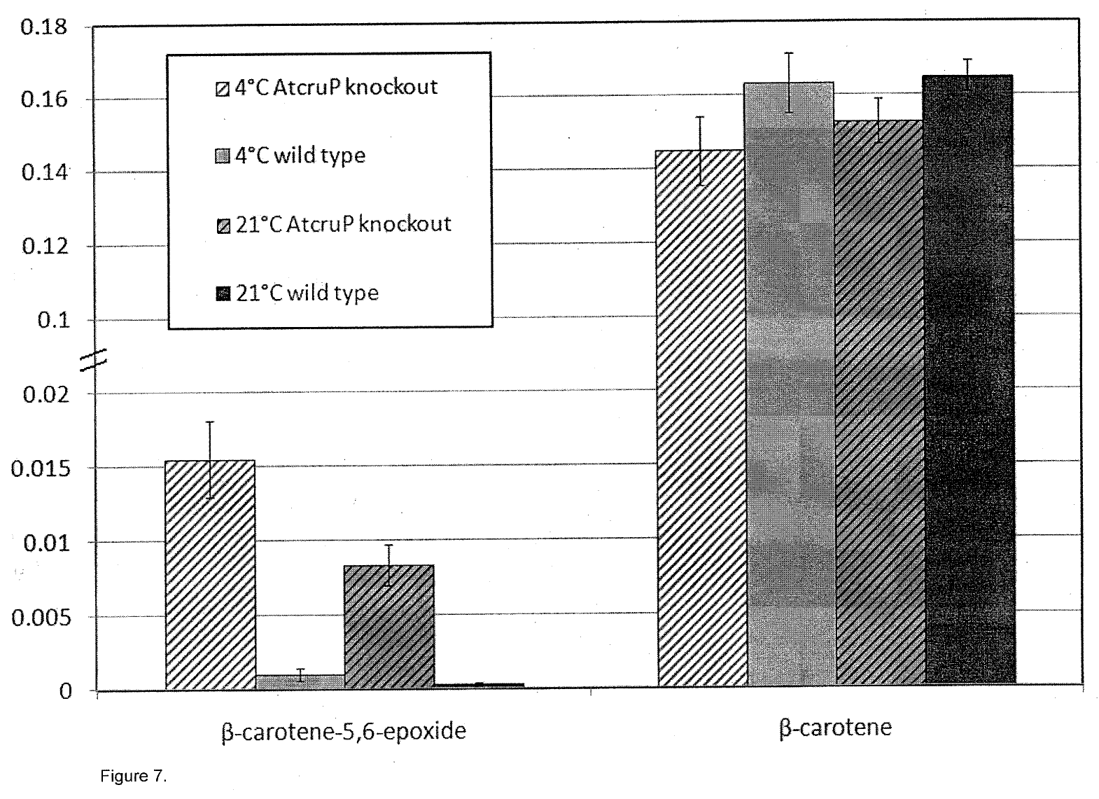

The retention time of the unknown peak was of a carotenoid that was more polar than α- and β-carotene but less polar than α- and β-cryptoxanthin (mono-hydroxylated carotenes). The spectrum (FIG. 5 E) suggested that this compound had fewer conjugated double bonds than β-carotene. A study of the literature for rare carotenoids found in plants provided many possibilities but none seemed more likely than β-carotene-5,6-epoxide, a carotenoid that has previously been observed in photosynthetic tissues. β-carotene-5,6-epoxide was synthesized (and subjected to HPLC analysis where it eluted at the same time and with the same spectrum as the unknown carotenoid (FIG. 6). Additional LC-MS analysis of the peak from the knockout plant confirmed a major ion fragment with a mass of 553 [M+H]⁺ corresponding to the mass of β-carotene-5,6-epoxide.

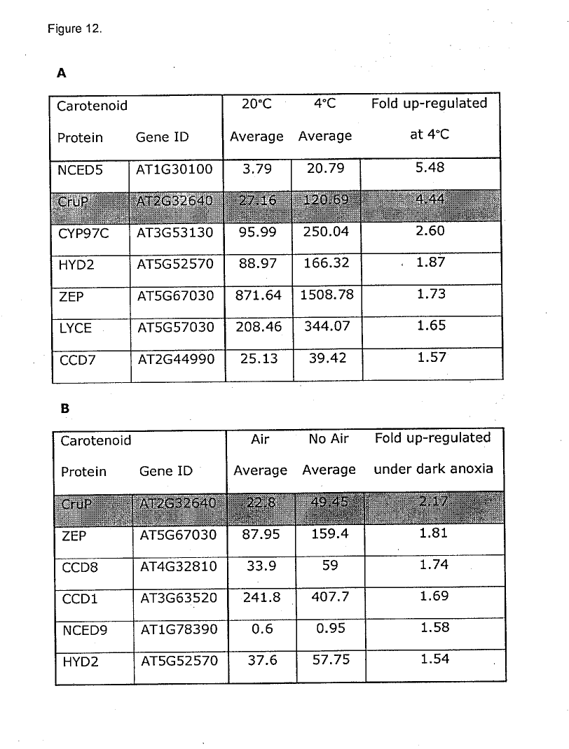

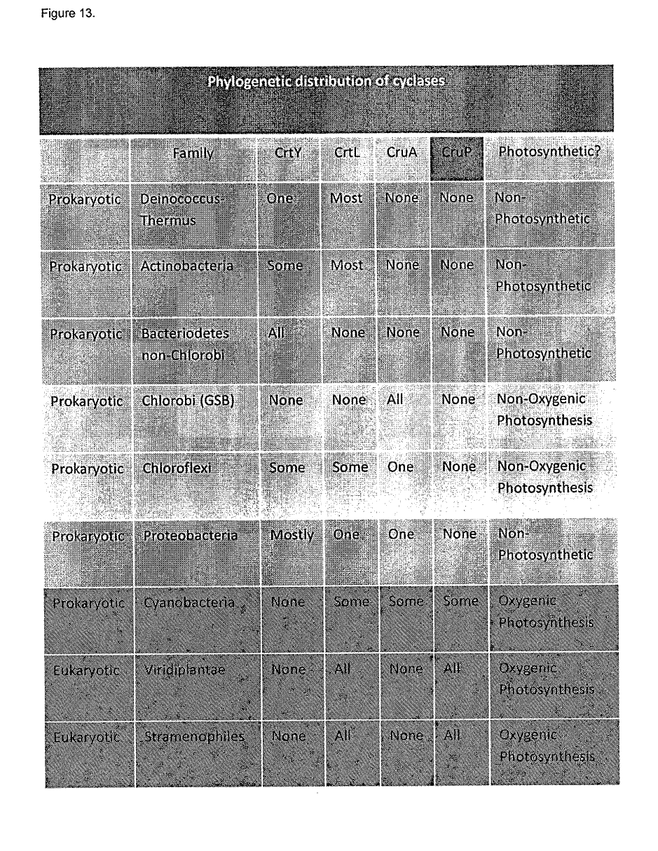

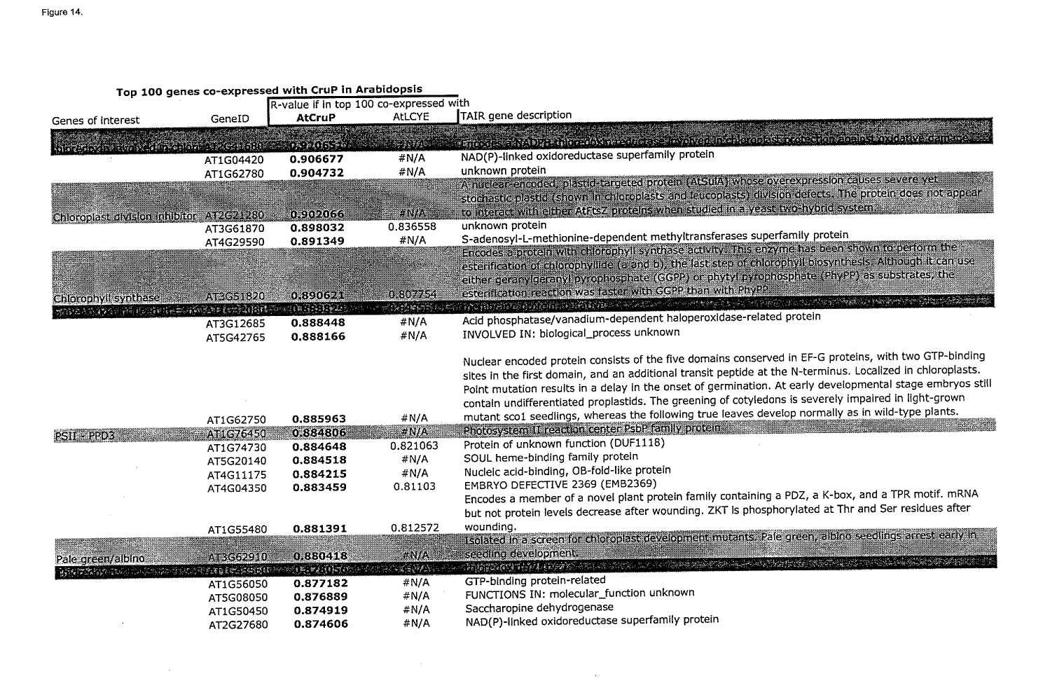

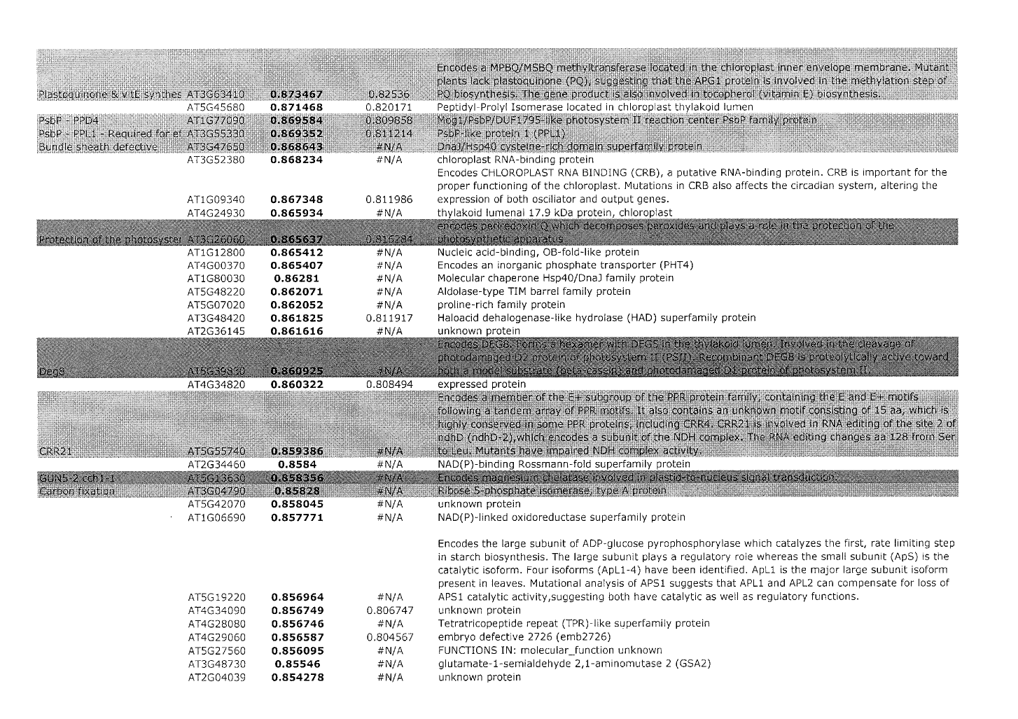

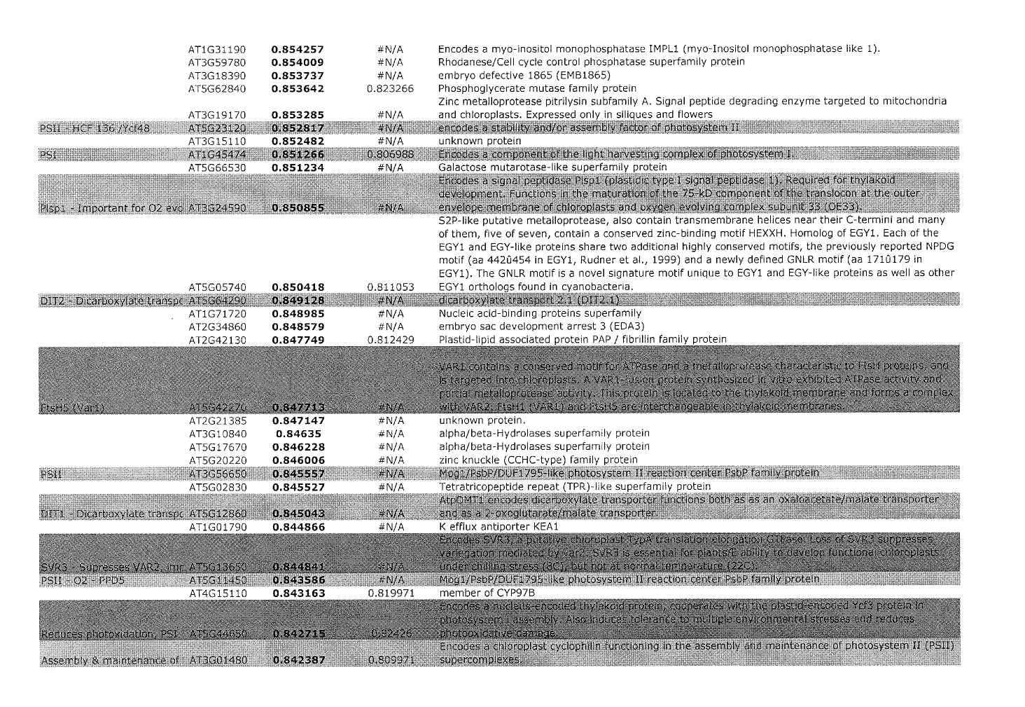

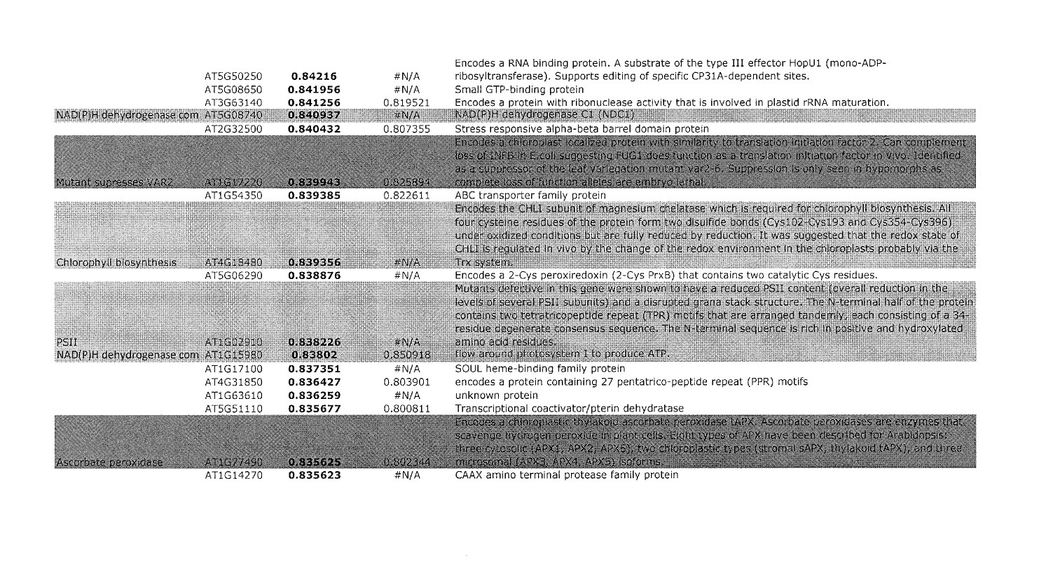

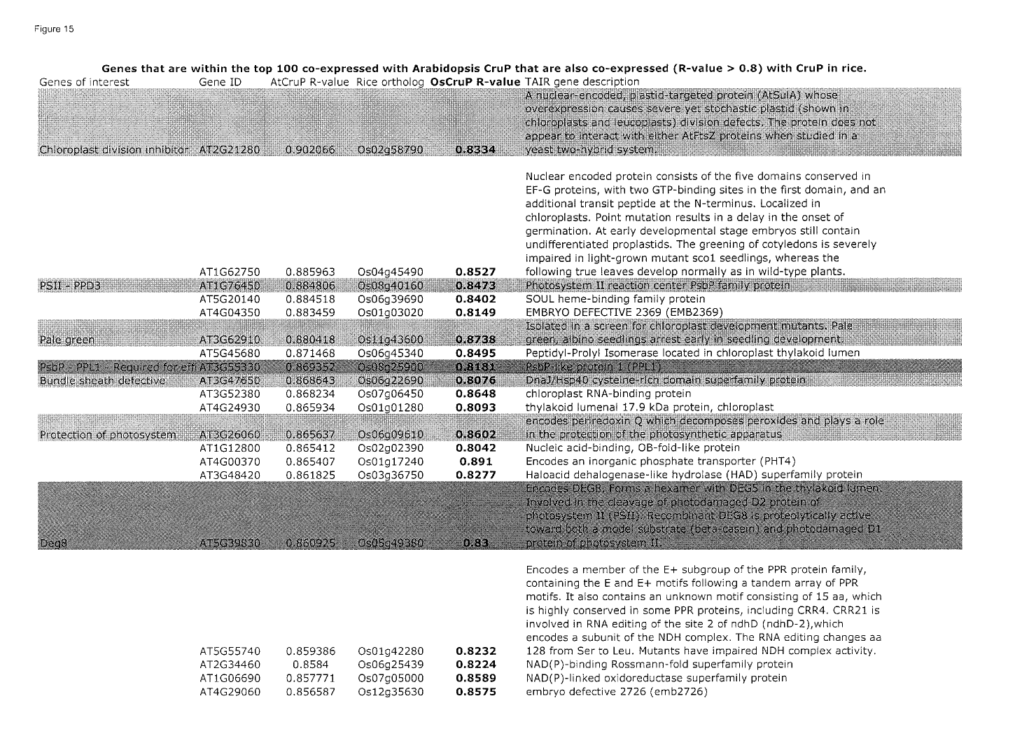

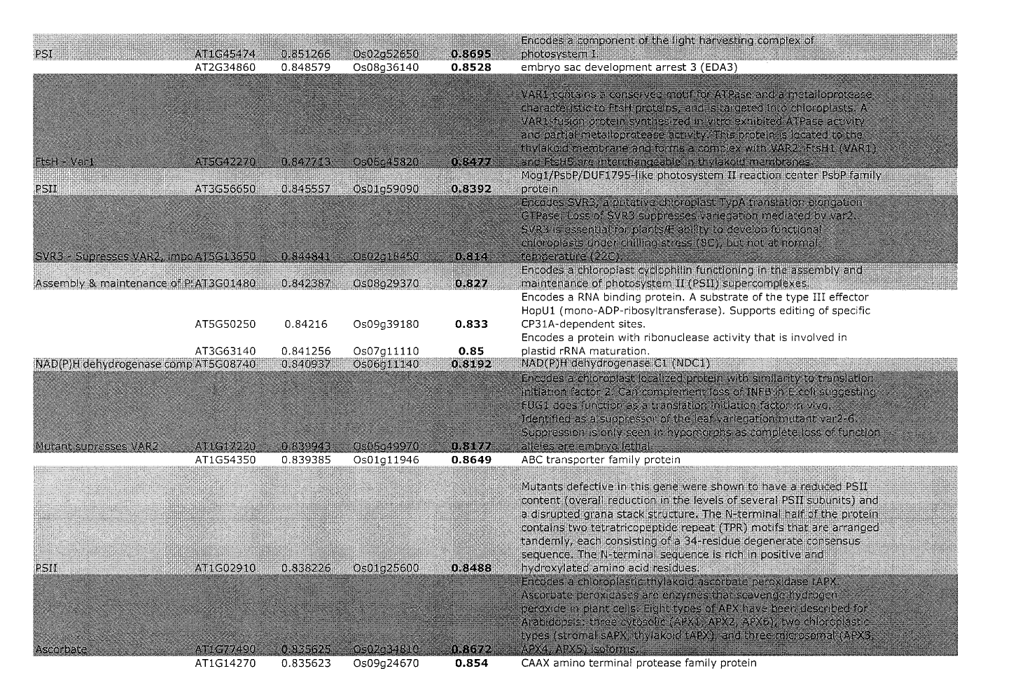

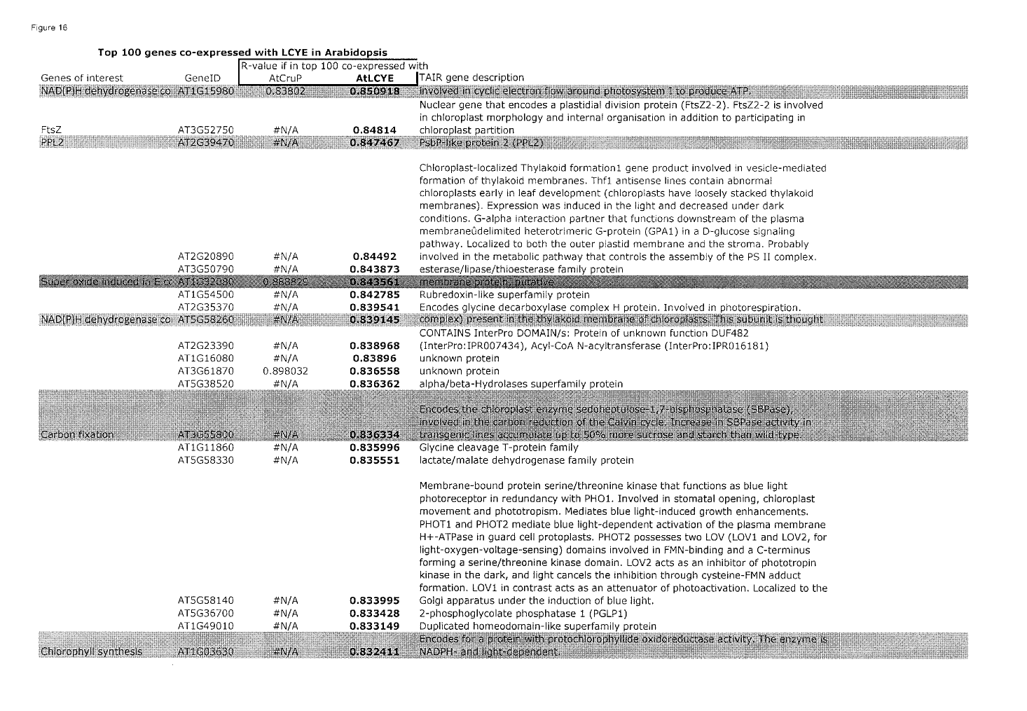

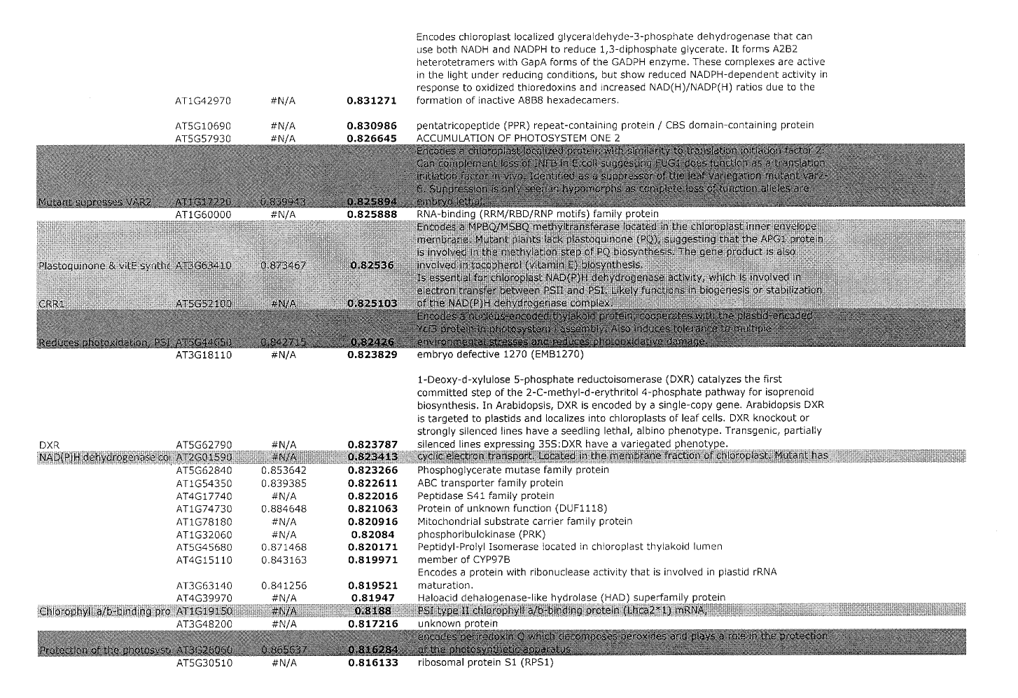

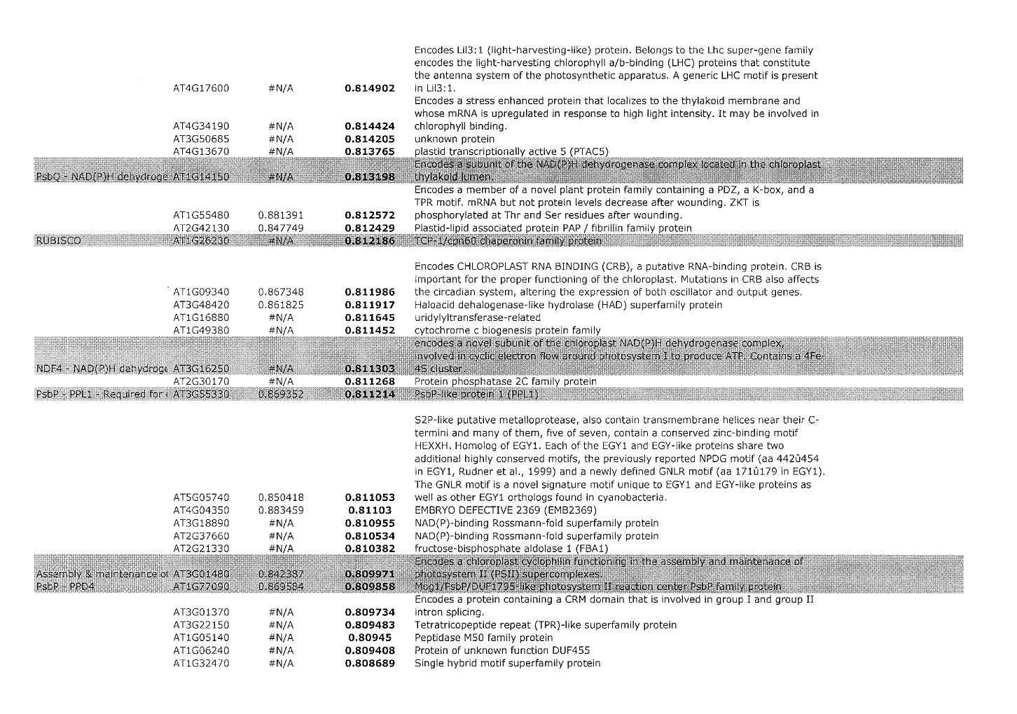

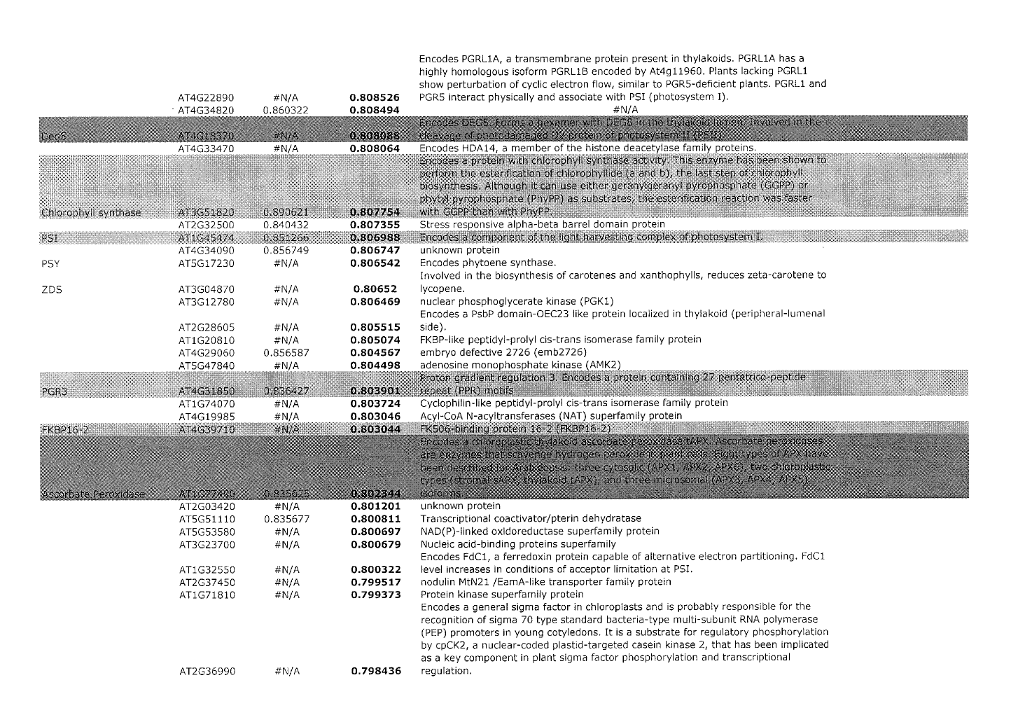

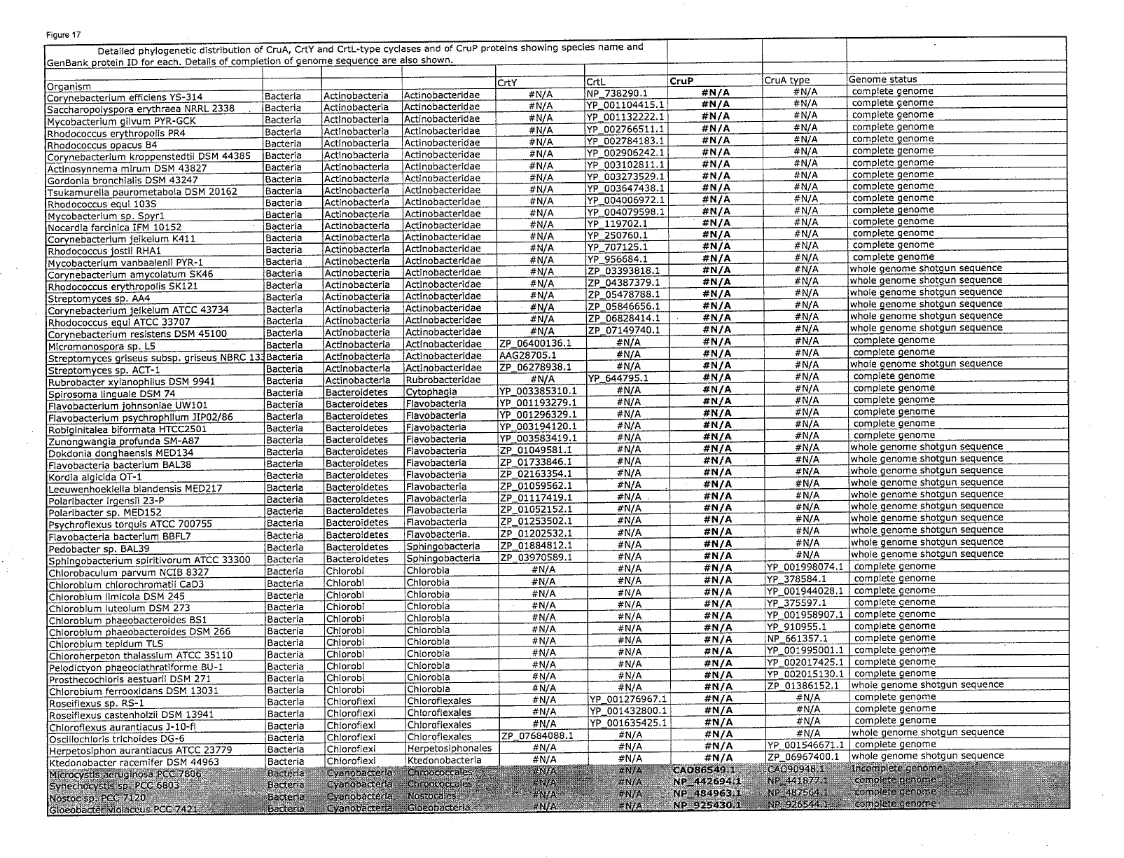

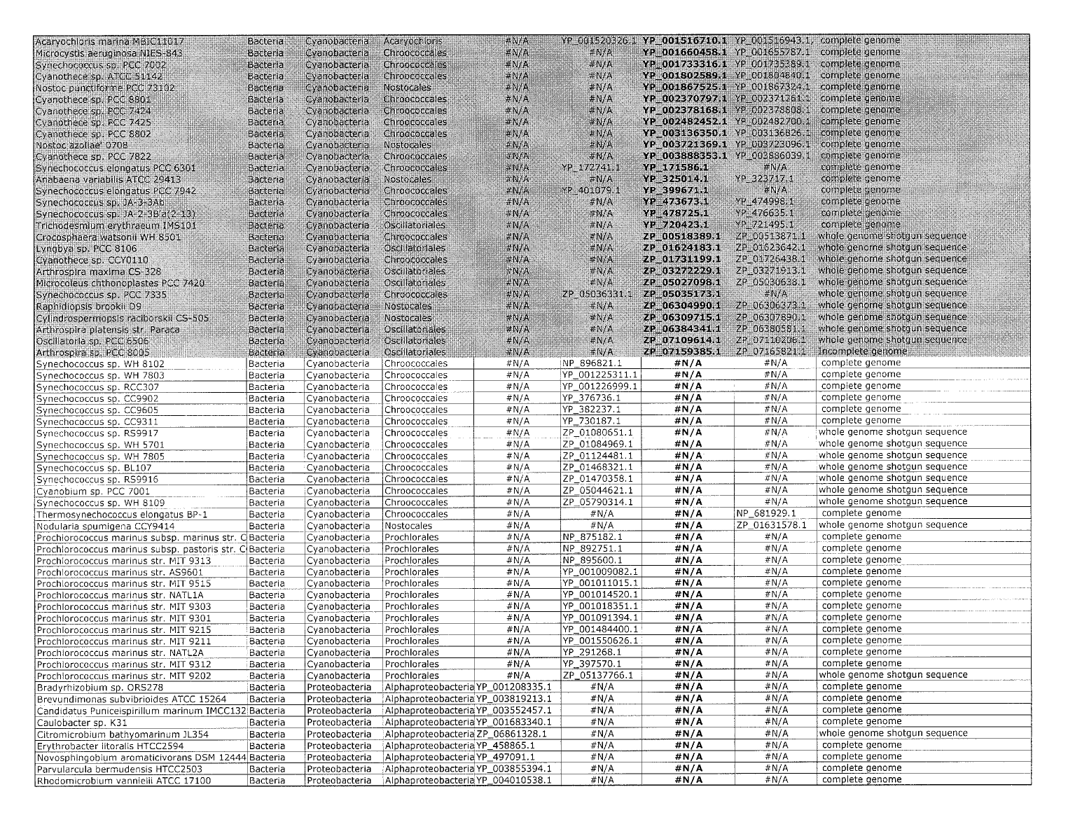

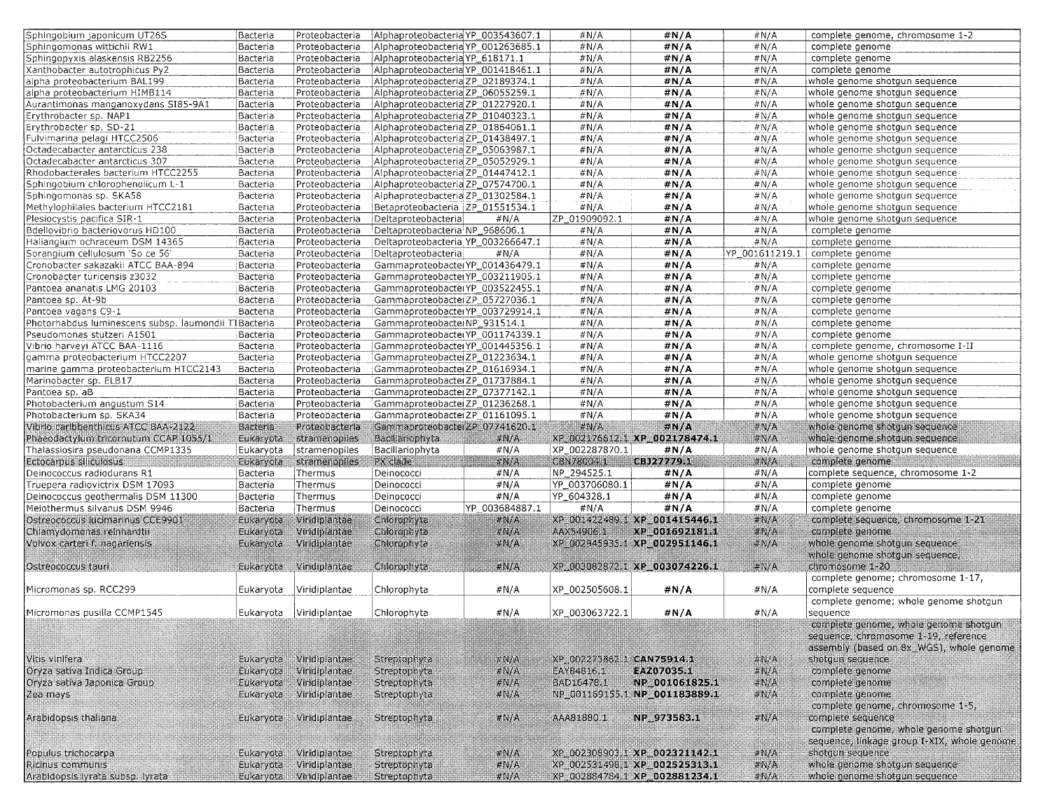

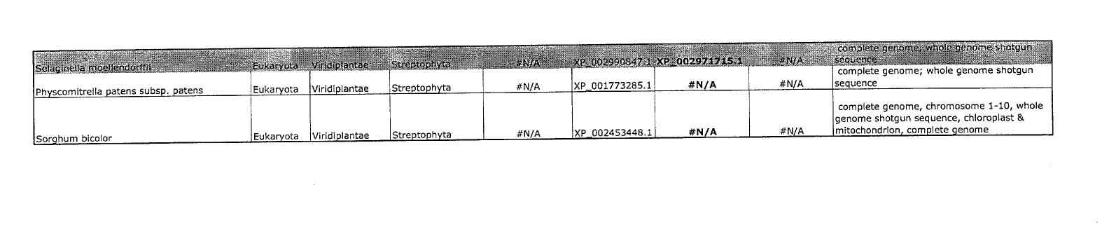

Since _{At}CruP transcripts are up-regulated at low temperatures (FIG. 12), _{At}CruP knockout plants were grown at 4° C. to observe the impact on levels of β-carotene-5,6-epoxide. Growth of both knockout and wild type plants at 4° C. for 10 days led to an increase in β-carotene-5,6-epoxide in both plants relative to plants grown at 21° C. (FIG. 7). A slight decrease in β-carotene was also observed in the _{At}CruP knockout in comparison to the wild type plant (FIG. 7). This decrease of β-carotene was approximately equivalent to the increase in β-carotene-5,6-epoxide. At 4° C. there was much more variation in β-carotene levels in both the _{At}CruP knockout and in the wild type but a decrease in β-carotene was still observed in the _{At}CruP knockout plants (FIG. 7).

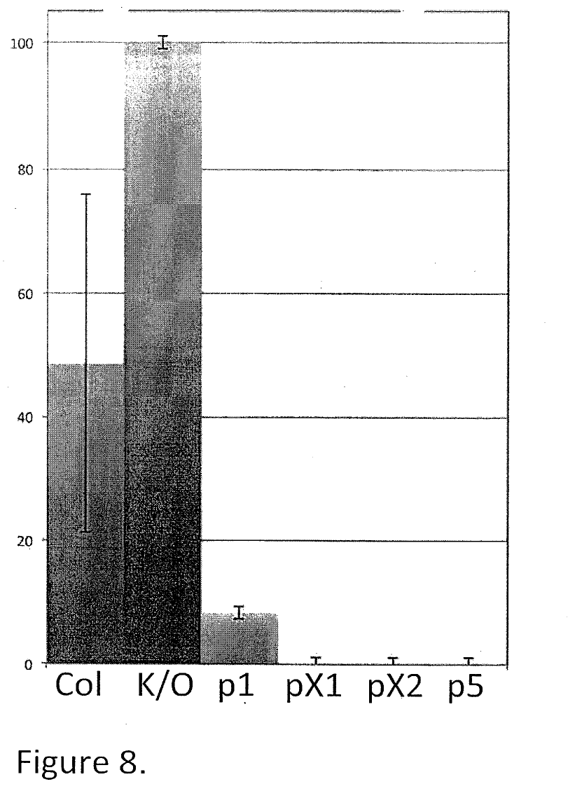

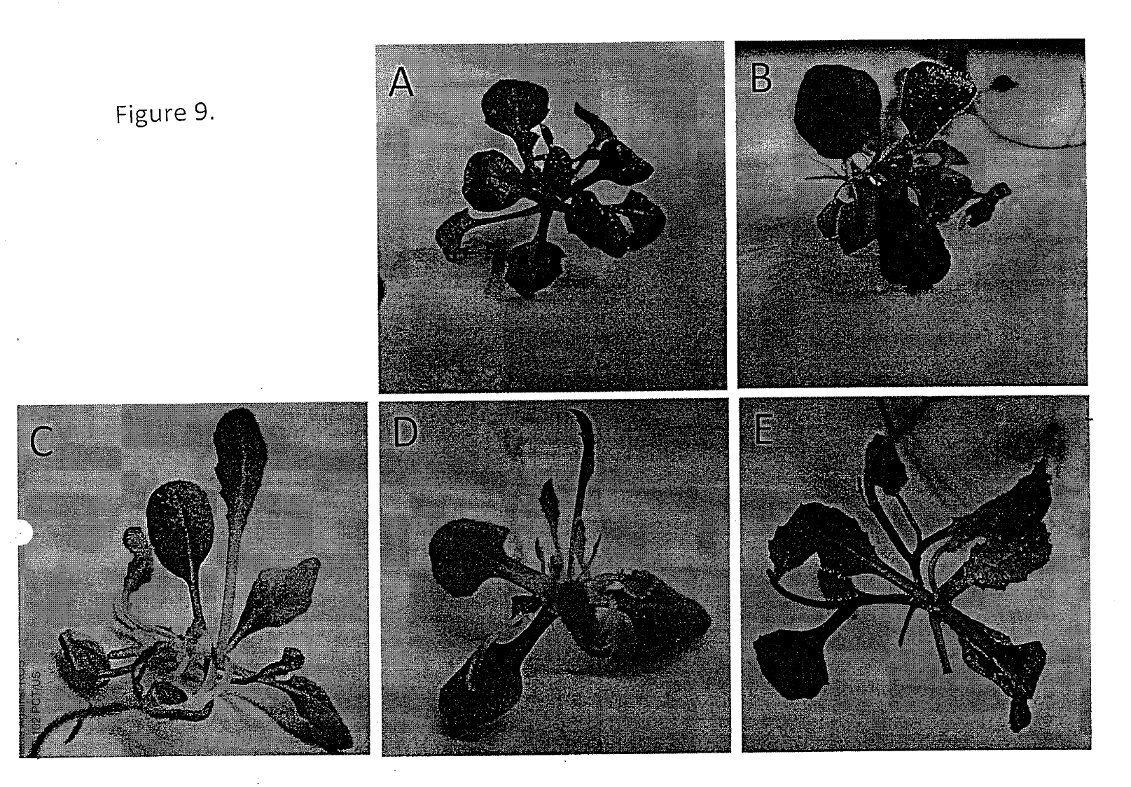

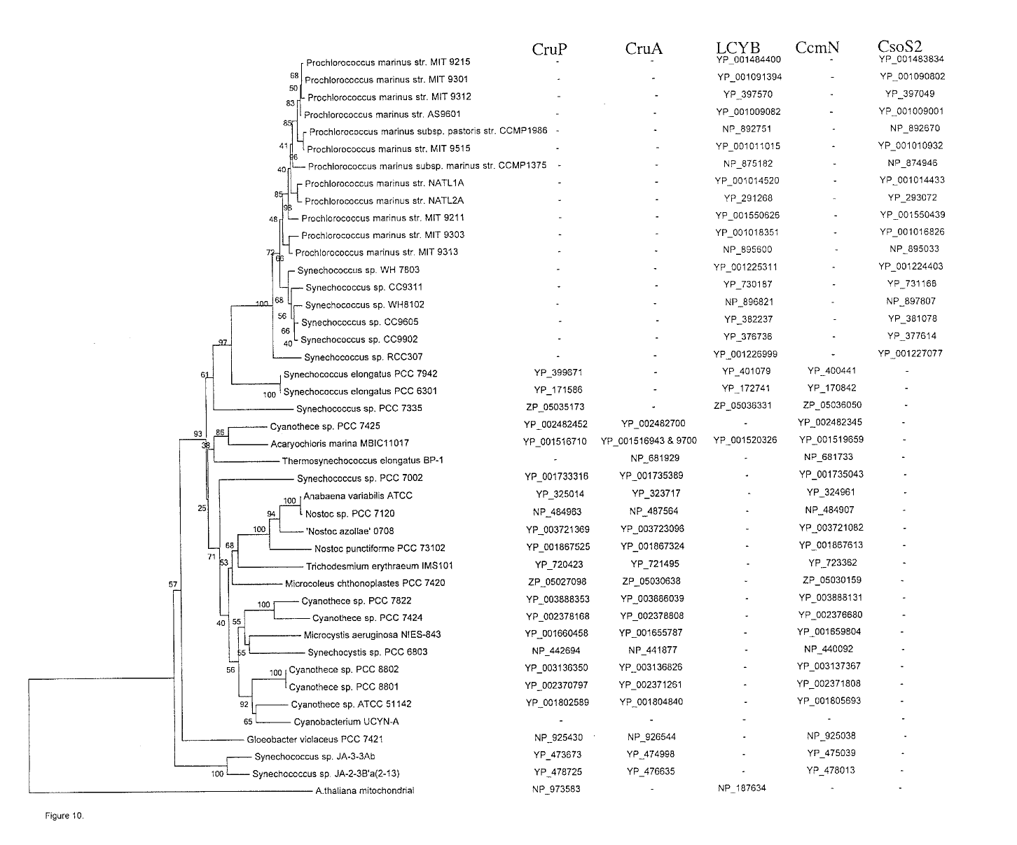

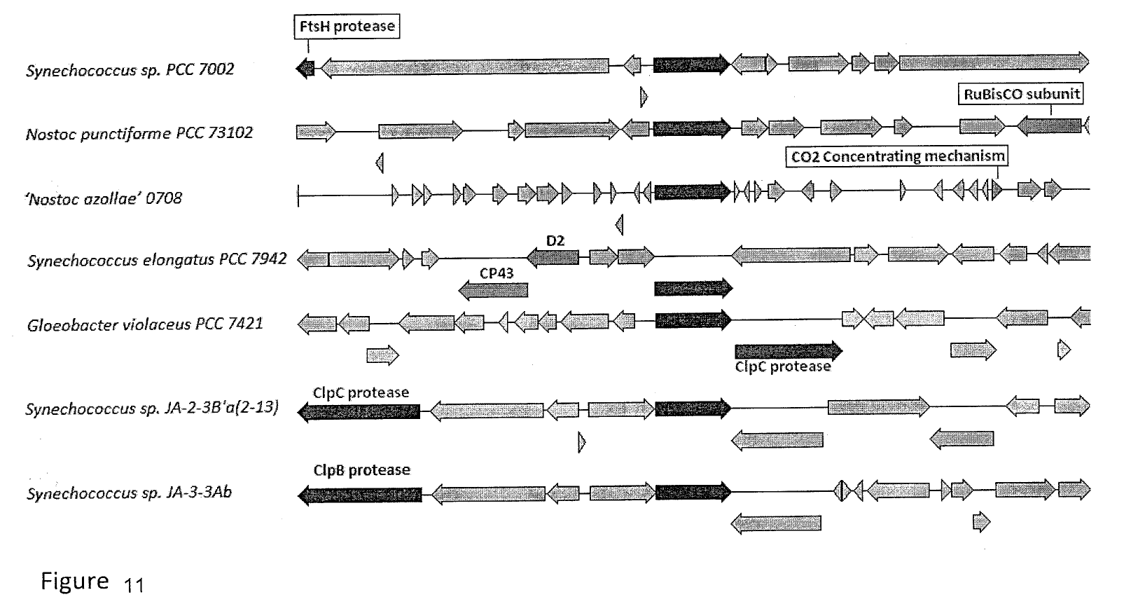

Considering published reports of β-carotene-5,6-epoxide formation via ROS mediated degradation of β-carotene in photosynthetic tissues exposed to high light stress, together with our own observations of β-carotene-5,6-epoxide accumulation in _{At}CruP knockout plants and up-regulation of CruP in ROS-producing conditions (cold stress and dark anoxic treatment), we considered that CruP may play a role in reducing the accumulation of ROS. Cotyledons from Columbia (wild type), _{At}CruP knockout and 35S:_{Zm}CruP lines exposed to anoxic conditions for one week were stained with nitrotetrazolium blue to screen for levels of ROS (FIG. 8). Wild type plants showed partial staining while _{At}CruP knockout plants showed staining throughout the entire cotyledon. 35S:_{Zm}CruP lines showed minimal to no staining in comparison to the wild type plants. These results clearly demonstrated that ROS levels in cotyledons are inversely correlated with CruP transcript levels. To further investigate the connection between CruP and stress-responses, wild type Columbia, _{At}CruP knockout and three 35S:_{Zm}CruP lines were grown for one month under standard conditions at 21° C. before being transferred to 4° C. with continuous light (50 μmol·m⁻²·s⁻¹) for two months. This cold stress treatment revealed a striking difference between plants that differed only in CruP levels. Columbia and _{At}CruP knockout plants developed deep anthocyanin staining throughout the entire plant whereas the three over-expressor 35S:_{Zm}CruP lines remained a deep green color with minimal or no anthocyanin development (FIG. 9). The anthocyanin response is consistent with increased ROS in the wild type and knockout plants and decreased ROS in the overexpression lines.

Co-expression analysis of genes encoding _{At}CruP and _{At}LCYE (FIG. 16) showed that the majority of co-expressed genes encode proteins involved in chlorophyll biosynthesis, photosystem repair or other photosynthesis related functions. Photosynthesis related genes that were co-expressed with _{At}CruP, but not _{At}LYCE, included many genes involved in protection of PSII against oxidative damage as well as those involved in repair of PSII after damage by singlet oxygen. Such genes include those encoding thioredoxins, HCF136, the PSII reaction center D1 proteases DEG8 and FtsH5 (also known as VAR1), SVR3—important for chloroplast development in cold conditions, mutants of which suppress variegation in var2 mutants and PPL1 involved in PSII repair. Other notable co-expressed genes include cch1-1 (gun5) involved in generating a putative plastid to nucleus signal in response to high light stress as well as genes encoding dicarboxylate transporters DIT1 & 2, mutants of which require high CO₂ for survival, a ribose 5-phosphate isomerase involved in CO₂ fixation and a YfhF homolog involved in inhibiting chloroplast division. Many of these genes were also co-expressed with the gene encoding CruP from Oryza sativa (_{Os}CruP). Chlorophyll related genes that were co-expressed with the gene encoding _{At}LCYE but not _{At}CruP include four genes encoding proteins that are part of the NAD(P)H dehydrogenase complex that is involved in cyclic electron transfer around PSI) as well as genes that encode proteins involved in chlorophyll synthesis (e.g. protochlorophyllide reductase) and chlorophyll binding proteins of PSI & II (e.g. chlorophyll A-B binding protein). Another gene of note is ftsZ, encoding a chloroplast division protein that functions antagonistically with the YfhF homolog mentioned above.

Previous reports of CruP showed distribution of the gene in higher plants and some cyanobacterial species. To determine how important CruP is for fitness in photosynthetic organisms, we performed a BLAST analysis to more thoroughly determine the distribution of CruP. A protein BLAST of CruP from Synechococcus sp. PCC 7002 revealed that CruP orthologs are only found in oxygenic photosynthetic organisms. These organisms encompass various families such as cyanobacteria, green and brown algae, diatoms, mosses and higher plants, including both monocots and dicots. An analysis of CruA orthologs showed that as well as being found in oxygenic cyanobacteria that lack CrtL type cyclases, CruA was also present in non-oxygenic organisms such as Chlorobi, Chloroflexi and deltaproteobacteria. A protein BLAST analysis of CrtL from the cyanobacterium Synechococcus elongatus PCC 6301 showed that CrtL orthologs were scattered throughout various species and are by no means isolated to oxygenic photosynthetic organisms, as in the case of CruP.

A phylogenetic tree (FIG. 10) was constructed using 16S rRNA sequences of fully sequenced cyanobacteria and mitochondrial 16S rRNA of A. thaliana as an outlier. The tree revealed that those cyanobacteria that do not contain CruP belong to a distinct clade. Further BLAST analysis was undertaken using CsoS2 and CcmN, proteins involved in CO₂ concentrating mechanisms of distinct cyanobacterial groups, known as α- and β-cyanobacteria respectively. The results revealed that β-cyanobacteria contain CruP while α-cyanobacteria do not. Two exceptions were noted, Thermosynechococcus elongatus BP-1 and cyanobacterium UCYN-A which are β-cyanobacteria but do not contain CruP. The reason for this presence or absence of CruP in the separate groups is likely due to the different environments inhabited by these organisms. Hints as to the precise environmental factor(s) that influence the presence or absence of CruP might be gleaned from T. elongatus and cyanobacterium UCYN-A, two β-cyanobacteria that do not contain CruP.

In bacteria, genes involved in similar processes are often found clustered in the genome. It was determined what genes tend to cluster with CruP in select cyanobacterial genomes to see if it could be inferred as a function of CruP. Analysis of genes that cluster with CruP revealed genes with functions similar to those of genes that are found to be co-expressed with _{At}CruP and _{Os}CruP. Examples include genes encoding proteins with roles related to PSII D1 degradation such as YP_{—}001733313, an FtsH5 homolog in Synechococcus sp. PCC 7002, and the ClpC (NP_{—}925429, YP_{—}478720) and ClpB (YP_{—}473669) proteases in Gloeobacter violaceus PCC 7421, Synechococcus sp. JA-2-3B′a(2-13) and Synechococcus sp. JA-3-3Ab (FIG. 11). The genes encoding the PSII reaction center proteins D2 (YP_{—}399674) and CP43 (YP_{—}399675) of Synechococcus elongatus PCC 7942 are also clustered near CruP, as are the genes encoding YP_{—}001867515, a RuBisCO small subunit protein in Nostoc punctiforme PCC 73102 and YP_{—}003721373, encoding a carbon dioxide concentrating mechanism protein known as CcmK in ‘Nostoc azollae’ 0708 (FIG. 11). Orthologs of many of the genes that cluster with CruP in different cyanobacteria were found co-expressed with both _{At}CruP and _{Os}CruP and are important in oxygenic photosynthetic organisms under cold stress and low CO₂, suggesting that CruP is involved in the same process in cyanobacteria as it is in plants.

It has been discovered that CruP is a chloroplast protein but does not exhibit lycopene cyclase activity. While the prior art has reported lycopene cyclase activity from _{Syn}CruP in E. coli, we were not able to replicate these results despite obtaining ample expression levels of the _{Syn}CruP protein. We were able to replicate cyclase activity of _{Cp}CruA. The finding that CruP is a peripheral thylakoid membrane protein in chloroplasts, suggested the possibility of another chloroplast-localized role.

β-carotene-5,6-epoxide in the pigment profile of Arabidopsis CruP knockout plants at levels substantially higher than those found in wild type or 35S:_{Zm}CruP plants has been surprisingly discovered. The increase of β-carotene-5,6-epoxide in the _{At}CruP knockout coincided with an approximate equivalent decrease in β-carotene levels (FIG. 7). The level of β-carotene-5,6-epoxide was observed to increase in the _{At}CruP knockout in response to chilling stress (FIG. 7), a condition known to up-regulate _{At}CruP transcripts (FIG. 12). β-carotene-5,6-epoxide has been identified in intact and isolated thylakoid membranes of spinach and T. elongatus. The level of β-carotene-5,6-epoxide in thylakoid membranes of spinach increased in proportion to light intensity. In a study of the protective role of β-carotene in photosystems, isolated bacteriochlorophyll and β-carotene dissolved in oxygenated acetone was exposed to light and chlorophyll molecules were observed to be protected at the expense of β-carotene. The first product formed in this reaction was β-carotene-5,6-epoxide followed by progressively more oxygenated β-carotene products. Oxidation of carotenoids by singlet oxygen is an unavoidable consequence of oxygenic photosynthesis. This oxidation is especially true of β-carotene which is the only carotenoid found in the core of PSII, the site of the water splitting/oxygen evolving complex. The main role of β-carotene in the reaction center is quenching of singlet oxygen. Bleaching of this β-carotene by singlet oxygen has been proposed to trigger turnover of the D1 protein in the PSII reaction center. We showed that absence of CruP was associated with increased ROS and increased β-carotene-5,6-epoxide, while overexpression of CruP was associated with reduced ROS and reduced β-carotene-5,6-epoxide (FIGS. 5 and 8). The impact of CruP over-expression on anthocyanin production, a known ROS response, in cold treated plants was quite striking in comparison to wild type and CruP knockout plants (FIG. 9). Wild type and CruP knockout plants both showed accumulation of large amounts of anthocyanins under this stress condition while the over-expressers remained green and healthy. Anthocyanin accumulation is a well characterized response of plants to increased ROS production, again showing the impact of CruP activity on ROS levels in plants treated under photoinhibitory conditions.

In silico analysis revealed A. thaliana CruP is up-regulated under chilling stress and dark anoxia (FIG. 14). Cotyledons and pedicels, where CruP transcripts were shown to be elevated, have been shown to undergo photo-inhibition stress and high singlet oxygen production under normal “non photo-inhibitory” conditions, in comparison to true leaves. Chilling stress in plants causes a range of physiological effects similar to those observed under high light stress. In addition, cold stress causes inhibition of the PSII D1 repair mechanism due to decreased membrane fluidity, leading to further increases of ROS. Dark anoxic treatment of plants leads to generation of nitric oxide and also, paradoxically, increased levels of ROS, including super oxide anions and hydrogen peroxide. Dark anoxia for prolonged periods causes peroxidation of lipid membranes. A recent analysis of the expression of all known carotenoid synthesis genes in Synechococcus sp. PCC 7002 showed that despite the fact that “transcript levels for genes encoding enzymes producing γ- and β-carotene from geranylgeranyl-pyrophosphate were generally much lower under anoxic conditions”, CruP is in fact up-regulated greater than tenfold under dark anoxic conditions and is up-regulated greater than threefold by low CO₂ conditions, whereas cruA is down-regulated two fold under both of these photoinhibitory/singlet oxygen producing conditions. All of these conditions and tissues, in which CruP transcript levels are elevated, are observed to have increased ROS production in comparison to true photosynthetic tissues and optimal growth conditions.

Co-expression analysis of _{At}CruP revealed that _{At}CruP was co-expressed with genes that code for proteins that function in the protection or repair of PSII from oxidative damage (e.g. the D1 proteases FtsH5 and DEG8) or proteins involved in prevention of photoinhibition (e.g. those involved in inorganic carbon transport and fixation as well as chloroplast development in cold conditions). Cyanobacterial CruP genes were clustered with genes of the PSII reaction center and with genes involved in the repair of oxidatively damaged PSII reaction center proteins (e.g. FtsH5, ClpC and ClpB) as well as those involved in carbon acquisition and fixation. This gene clustering pattern fits with the observation that CruP transcripts are up-regulated in both Arabidopsis and Synechococcus sp. PCC 7002 under photoinhibitory/ROS producing conditions such as low CO₂ and chilling stress. In contrast, the gene encoding _{At}LCYE was co-expressed with genes involved in chlorophyll synthesis and photosystem assembly as well as ftsZ, a gene involved in chloroplast division that functions antagonistically with yfhF (a gene co-expressed with CruP) (FIG. 16). The observed co-expression pattern suggests differing roles for LCYE and CruP in growth and protection from oxidative damage, respectively.

While all other lycopene cyclases are found in a wide variety of organisms, both oxygenic and non-oxygenic phototrophs as well as non-photosynthetic organisms, CruP was only found in oxygenic phototrophs and only in conjunction with another lycopene β-cyclase. CruP was never found as the sole cyclase of any organism, whereas non-oxygenic phototrophs and non-phototrophs, as well as a few cyanobacteria, have only one lycopene cyclase (FIG. 17). This phylogenetic distribution of CruP, in comparison to other lycopene cyclases, suggests that either oxygenic photosynthesis has a requirement for more than one lycopene β-cyclase or that CruP has a function other than that of lycopene cyclization. The former hypothesis, that oxygenic photosynthetic organisms require more than one lycopene β-cyclase, seems unlikely since many cyanobacterial species have only one lycopene β-cyclase (either CrtL or CruA). Furthermore, this hypothesis does not explain the exclusion of CruP from all carotenoid producing non-oxygenic organisms.

A phylogenetic tree was constructed based on 16S rRNA from fully sequenced cyanobacteria (FIG. 10). This tree identified evolutionarily distinct clades of cyanobacteria. One clade contained cyanobacteria with CruP, while another distinctly separate clade contained cyanobacteria that lacked CruP. The top clade (FIG. 10) was populated by open ocean cyanobacteria, encompassing all but two of the non-CruP containing cyanobacteria. These open ocean cyanobacteria are also known as α-cyanobacteria which are characterized by having a CO₂ concentrating mechanism involving a CsoS2 protein that is not found in β-cyanobacteria. Those cyanobacteria that contain CruP (bottom clade in FIG. 10) are from diverse ecological habitats including fresh water, salt lakes, intertidal zones, hot springs, dry rocks, symbiotic relationships etc. These CruP containing cyanobacteria are known as β-cyanobacteria contain CO2 concentrating mechanisms that utilize a CcmN protein not found in α-cyanobacteria. Cyanobacteria are able to exchange genetic material via conjugation, and would therefore retain CruP in the genome if it provided an evolutionary advantage. This distribution suggests CruP provides increased fitness to most β-cyanobacteria but not to α-cyanobacteria. β-cyanobacteria are exposed to variety of environmental extremes, in particular temperature fluctuations (including chilling stress) and inorganic carbon limitations are two environmental conditions that β-cyanobacteria have to deal with but α-cyanobacteria do not. Chilling stress and low inorganic carbon are both conditions that lead to photoinhibition and to the up-regulation of CruP transcripts. Two cyanobacteria were noted as exceptions to the β-cyanobacterial distribution of CruP, T. elongatus and cyanobacterium UCYN-A are both β-cyanobacteria that lack CruP. T. elongatus is a thermophilic cyanobacterium isolated from Beppu hot springs in Japan and this cyanobacterium reportedly has a reduced set of inorganic carbon transporters in comparison to other fresh water cyanobacteria. It is likely that the waters at this hot spring contain high levels of inorganic carbon due to mixing of volcanic CO₂ as has been reported for nearby thermal springs. As such, this cyanobacterium would not experience cold stress or inorganic carbon limitations in its natural environment, explaining the absence of CruP in this organism. Cyanobacterium UCYN-A is an unusual cyanobacterium with a reduced genome and no genes encoding PSII complex proteins or carbon fixation enzymes. Blast analysis revealed no genes with homology to CrtL, CruA or CruP in the complete genome of this organism (FIG. 10), suggesting another class of lycopene cyclase may exist and adding further evidence that CruP is not required in the absence of PSII, i.e. in non-oxygenic photosynthetic organisms.

In conclusion, as a result of the herein invention, it has been shown that absence of CruP is associated with increased ROS and increased β-carotene-5,6-epoxide, while overexpression of CruP is associated with reduced ROS, reduced β-carotene-5,6-epoxide and a significantly reduced anthocyanin response under cold stress. The above results suggest that the function of CruP is to reduce oxidative damage caused by singlet oxygen. The above conclusion explains the presence of β-carotene-5,6-epoxide, the anthocyanin response (or lack thereof in over-expressors) observed in cold treated plants (FIG. 9) and slow growth of the Arabidopsis mutant as well as the disordered thylakoid structure of the Synechocystis CruP knockout. ROS generated by high excitation pressure of the photosystems during early development can cause a failure of chloroplasts to assemble organized internal structures. The lack of an observable difference in the pigment profile of cyanobacterial CruP knockouts combined with the lack of lycopene cyclase activity of _{Syn}CruP in our study and the limited phylogenetic distribution of CruP, strongly suggest that CruP has a function other than lycopene cyclization. Considering the consistently observed up-regulation of CruP transcripts to photoinhibitory, ROS producing conditions, the limited phylogenetic distribution of CruP and the inverse association between CruP transcript levels and ROS levels (and chemical markers of ROS levels), it appears that CruP plays a role, directly or indirectly, in reducing ROS levels in oxygenic photosynthetic organisms under photoinhibitory stress. Thus, CruP represents a new target for developing resilient plants and algae needed to supply food and biofuels in the face of global climate change. The up-regulation of CruP during cold and anoxic conditions, such as flooding, suggests also that CruP will be an important locus to consider in screening for cold and submergence (anoxia) tolerance in plants.

### Examples

### AtCruP Knockout and 35S: _{Zm}CruP Lines

An Arabidopsis thaliana CruP knockout line (SALK_{—}011725) carrying a T-DNA insert in the CruP gene in the Columbia background was obtained from The Arabidopsis Information Resource (TAIR). Real-time PCR was performed to confirm absence of CruP transcripts in the knockout line as described below. For the generation of 35S:_{Zm}CruP over-expressing A. thaliana plants, Agrobacterium tumefaciens strain GV3101 (pMP90) was transformed with pRed-_{Zm}CruP using the freeze thaw method and selected using 50 μg/ml gentamicin and 50 μg/ml kanamycin. Floral dip transformation of A. thaliana was performed. See below. Transgenic seeds were selected using a pair of red-lens sunglasses (KD's Dark Red, http://www.originalkds.com). Seeds that glowed red under a light of wavelength 550 to 560 nm were used in this study for over-expression experiments. Real-time PCR was performed to confirm over-expression as described previously. Primers 2617 & 2618 (Table 1) were used for amplification of actin cDNA, 1871 & 2190 (Table 1) for amplification of _{Zm}CruP cDNA and 2978 & 2979 (Table 1) for amplification of _{At}CruP cDNA. See below.

### Standard Plant Growth Conditions

Unless otherwise stated, plants were grown in a Percival Scientific growth chamber under a 16 hr day, 8 hr night cycle with a light intensity of 50 μmol·m⁻²·s⁻¹ and a constant temperature of 21° C. Plants were watered every four to seven days.

### Pigment Extraction & Analysis

Epoxy-5,6-β-carotene was synthesized according Barua (see below). Plant carotenoids were extracted by grinding roughly 30 mg of four week old plant tissue in 500 μl of 60:40 acetone:ethyl acetate, 400 μl of H₂O was added before vortexing and centrifugation for 5 min at 17,000 g. The upper ethyl acetate fraction was washed, spun at 17,000 g for 5 min then transferred to a new tube and dried under nitrogen before re-suspension in methanol for HPLC analysis.

Separation of carotenoid and chlorophyll pigments was carried out using a Waters HPLC system equipped with a 2695 Alliance separation module, a 996 photodiode array detector, a Develosil C₃₀ RP-Aqueous (5 μm, 250×4.6 mm) column (Phenomenex, Torrance, Calif.), and a Nucleosil C₁₈ (5 μm, 4×3.0 mm) guard column (Phenomenex, Torrance, Calif.). Solvent A consisted of acetonitrile:methanol:H₂O (84:2:14) and solvent B consisted of methanol:ethyl acetate (68:32). Initial flow conditions consisted of 100% A at a flow rate of 0.6 ml/min. Using a linear gradient, flow was changed to 100% B by the 60 min mark at this point flow rate was increased to 1.2 ml/min and held for an additional 50 min before being re-equilibrated with A for 5 min. Column temperature was held at 30° C. and 100 μl of each sample was injected for pigment analysis.

LC-MS was performed on a Waters 2695 HPLC equipped with a 2998 PDA detector coupled to a Waters LCT Premiere XE Time of Flight (TOF) Mass Spectrometer system using electrospray ionization in positive ion mode.

### Chloroplast Isolation and Protein Import:

_{Zm}CruP was transcribed and translated in vitro from pTNT-_{Zm}CruP-StrepTag using SP6 polymerase in rabbit retoculocyte lysate TnT coupled system (Promega) in the presence of ³⁵5-methionine (PerkinElmer). Pea (Pisum sativum, var. Green Arrow, Jung Seed) plants were grown in a growth chamber, at 18-20° C., 14/10 h dark/light cycle at 425 μmol m⁻² s⁻¹. Plants were harvested and used for chloroplast isolation after 10-14 days as described previously (58) (see below).

### Protoplast Isolation and Transient Expression:

Maize (Zea mays var. B73) mesophyll protoplasts were isolated from 10 day old second leaves and transfected with the pUC35S-_{Zm}CruP-GFP vector, encoding a _{Zm}CruP-GFP fusion protein, by PEG-mediated transformation (see below).

### Phylogenetic/in-Silico Analysis:

The Arabidopsis Co-expression Data Mining Tools (ACT) website was used for analysis of genes that were co-expressed with _{At}CruP (At2g32640) and the gene encoding _{At}LCYE (At5g57030). The Rice Oligonucleotide Array Database website was used for analysis of genes that were co-expressed with _{Os}CruP (Os8g32630). Genevestigator was used for analyzing variation of _{At}CruP transcript levels under different conditions and in various tissues. The SEED database was used for analysis of genes that clustered with cyanobacterial CruP genes.

The results of a protein BLAST search using the Synechococcus sp. PCC 7002 CruP protein sequence were compared to the results of a protein BLAST of the Synechococcus sp. PCC 7002 CruA protein sequence. Only results with an e-value greater than 0.005 (the standard BLAST cut-off score) were used. Those proteins that had a smaller E-value in the CruP set were considered CruP orthologs; the others were considered CruA orthologs.

16S rRNA sequences from cyanobacteria with complete genomes were obtained from NCBI genomes (http://www.ncbi.nlm.nih.gov/genome) aligned using ClustalW2 (European Bioinformatics Institute, http://www.ebi.ac.uk/Tools/msa/clustalw2/). Alignments were then imported into MEGA 5.05 (65) for construction of a neighbor joining tree with 1000 replications for bootstrap values.

### ROS Analysis

Seeds were sterilized in 1 ml 70% ethanol for 5 min followed by 5 min in 1 ml 25% bleach solution containing 0.01% Tween100. The bleach solution was removed and seeds were rinsed five times with sterile milliQ H₂O. The seeds were soaked under 900 μl of sterile milliQ H₂O (to create a photo-inhibitory environment) and placed at 4° C. for two days before being placed at 21° C., 14 hrs light, 10 hrs dark for 2 weeks. Plants were stained with 2 mM nitrotetrazolium blue (NTB) in 20 mM phosphate buffer (pH 6.1) for 15 min. Reactions were stopped by removing the NTB solution and flushing with sterile distilled water.

Plasmids Used in this Study:

Zea mays CruP (_{Zm}CruP) cDNA clone (clone ID: ZM_BFc0139E12) pSPORT-_{Zm}CruP was ordered from the Arizona Genomics Institute. Using BamHI and EcoRI the gene encoding GFP was cut from pBIG121 and cloned into the same sites of pUC35S-GUS-Nos to generate pUC35S-sGFP-Nos. _{Zm}CruP was PCR amplified from pSPORT-_{Zm}CruP using primers 2487 and 2938 (Table 1) to incorporate XbaI and BamHI restriction enzyme sites respectively. The PCR product and pUC35S-sGFP-Nos were digested with XbaI and BamHI before ligation to generate the plasmid pUC35S-_{Zm}CruP-GFP for use in transient expression studies. For protein import studies ZmCruP was amplified from pSPORT-_{Zm}CruP using the primers 2960 & 2958, digested with XhoI & XbaI and inserted into these sites of the pTNT vector (Promega) giving the vector pTNT-_{Zm}CruP-StrepTag. _{Zm}CruP was PCR amplified from pSPORT-_{Zm}CruP using primers 2487 and 2930 (Table 1) to incorporate two XbaI restriction enzyme sites. The PCR product and pBin35SRed, containing a 35S promoter to drive expression of the transgene, were digested with XbaI before ligation to generate the plasmid pRed-_{Zm}CruP for use in Arabidopsis stable transformation. The _{Cp}CruA plasmid (p16-CPL1) was used as a positive control. Synechococcus sp. PCC 7002 CruP (_{Syn}CruP) ORF (NC_{—}010475) was synthesized and cloned into pUC57 by GenScript to generate pUC-_{Syn}CruP. _{Syn}CruP was PCR amplified from pUC-_{Syn}CruP using primers 2986 & 2987 (Table 1) to incorporate an NdeI site upstream of the start codon and a BamHI site downstream of the stop codon. After digestion of this PCR product and of pET16b (Novagen) with these restriction enzymes, _{Syn}CruP was ligated into pET16b to generate pET-_{Syn}CruP, used for in vitro studies.

Bacterial Complementation and Pigment Analysis:

For carotenoid analyses, saturated cultures in LB medium were diluted 100 fold into 50 ml fresh medium in 250 ml flasks, grown in the dark at 250 rpm at 37° C. until OD₆₀₀ 0.5, at which point they were induced with 5 mM IPTG and further cultured for a total of three days at 28° C. For extraction of carotenoids produced in bacteria, bacterial cultures were centrifuged at 2,000 g for 10 min at 4° C., the supernatant was removed and 5 ml of 50:50 methanol:acetone added to the bacterial pellet before vigorously vortexing. The sample was again centrifuged at 2,000 g for 10 min at 4° C. and the supernatant transferred to a new tube. The sample was dried down under nitrogen and re-suspended in 500 μl of 60:40 acetone:ethyl acetate, 400 μl of H₂O was added before vortexing and centrifugation for 5 min at 17,000 g. The upper ethyl acetate fraction was transferred to a new tube, spun at 17,000 g for 5 min and again transferred to another tube before being dried down under nitrogen and re-suspended in methanol for HPLC analysis.

Generation of 35S:_{Zm}CruP Lines Segregating Populations:

Agrobacterium tumefaciens strain GV3101 (pMP90) was transformed with pRed-_{Zm}CruP using the freeze thaw method and selected using 50 μg/ml gentamicin and 50 μg/ml kanamycin. Floral dip transformation of A. thaliana was performed according to as described below. A single Agrobacterium colony was grown overnight in 20 ml LB with 50 μg/ml gentamicin and 50 μg/ml kanamycin. 4 ml of overnight culture was transferred to 400 ml LB and grown to OD₆₀₀ 0.8. Agrobacterium cells were pelleted by centrifugation at 1500 g at 4° C. for 15 min before being re-suspended in 700 ml 5% sucrose+0.05% silwet 77 (Lehle Seeds). Plant flowers were soaked in the Agrobacterium solution for 45 sec then placed in a tray lined with wet paper towels & covered to maintain high humidity and placed in the dark for 12-16 hrs. Plants were taken out to air dry before returning them to standard growth conditions (see below). Plants were watered for 3 weeks then left to dry out before seeds were collected. Segregating populations were generated by transferring pollen from _{At}CruP knockout plants to the stigma of wild type Columbia plants and also by transferring pollen from _{At}CruP knockout plants to the stigma of 35S:_{Zm}CruP plants. Primers 2292 & 2293 (Table 1) were used to screen for plants with no T-DNA insert in the _{At}CruP gene and the primers 2292 & 1857 (Table 1) were used to screen for the presence of a T-DNA insert of the _{At}CruP gene. Transgenic 35S:_{Zm}CruP over-expressing plants were selected at the seed stage as described above.

Real-Time PCR:

For real-time PCR, total RNA was isolated from Arabidopsis lines using the RNeasy Plant Mini Kit (Qiagen), then treated with DNase I according to the manufacturer's instructions (Invitrogen). First strand cDNA synthesis was carried out using an oligo (dT) primer and Superscript III RT (Invitrogen) according to the manufacturer's instructions. 10 ng of cDNA was used for each real-time PCR and samples were amplified using a SYBR GreenER Supermix (Bio-Rad). Thermal cycling conditions in a MyIQ Single-Color Real-Time PCR detection system (Bio-Rad) included an initial incubation at 94° C. for 10 sec, followed by 35 cycles of 95° C. for 10 sec, 58° C. for 35 sec, and 72° C. 10 sec. The relative quantity of the transcripts was calculated using the comparative threshold cycle (CT) method. Actin2 (AT3G18780) was used as a standard for normalization between samples. Three technical replicates of each experiment were performed.

β-Carotene-5,6-Epoxide Synthesis:

β-carotene-5,6-epoxide was synthesized according to. β-carotene was mixed with 0.3× molar concentrations of M-Chloroperbenzoic acid in diethyl ether and stirred at 4° C. for 1 hr. This solution was then mixed with equal parts water containing dilute NaOH before centrifugation at 17,000 g for 5 min. The top diethyl ether fraction was transferred to a new tube, washed with water, spun and the diethyl ether fraction was transferred to a new tube before being dried down under nitrogen and re-dissolved in methanol for HPLC analysis.

Chloroplast Isolation and Protein Import:

Pea (Pisum sativum, var. Green Arrow, Jung Seed) plants were grown in a growth chamber, at 18-20° C., 14/10 h dark/light cycle at 425 μmol m⁻² s⁻¹. Plants were harvested and used for chloroplast isolation after 10-14 days as described previously. To prevent starch accumulation, plant leaves were collected after 8 hours of dark, and then grinded with a kitchen blender in a grinding buffer (50 mM HEPES pH=8, 330 mM sorbitol, 1 mM MnCl₂, 1 mM MgCl₂, 2 mM EDTA, 0.2% w/v BSA, 0.1% w/v ascorbic acid) at 4° C. Chloroplasts were isolated using Percoll gradient centrifugation. The Percoll gradient was prepared by centrifugation of 50% Percoll (Sigma) in the grinding buffer (40,000 g, 30 min, 4° C.). Chloroplast suspension was layered on the top of the gradient and centrifuged at 12,000 g for 10 min. The lower band, containing intact chloroplasts, was aspirated and washed with the import buffer (50 mM HEPES pH=8, 330 mM sorbitol). Intact chloroplasts were resuspended in 140 μl/reaction of import mix (50 mM HEPES pH=8, 330 mM sorbitol, 4 mM methionine, 4 mM ATP, 4 mM MgCl₂, 10 mM K—Ac, 10 mM NaHCO₃) at a concentration 50 μg of chlorophyll/reaction. For the import reaction 10 μl of in vitro transcription/translation product were added to the chloroplast mix. After 25 min, at 25° C. in the light, the import reaction was stopped by placing on ice and diluting with 500 μl of cold import buffer, chloroplasts were collected by centrifugation (800 g, 2 min), diluted in 200 μl of fresh cold import buffer, supplemented with 1 mM CaCl₂, and divided into two parts, one was left intact, the other was treated with 125 ng/μl of thermolysin for 30 min on ice. The reaction was stopped by adding EDTA to a final concentration of 10 mM. Chloroplasts were collected by centrifugation at 800 g for 2 min. Sample buffer for SDS-PAGE was added to the pellets and the protein extracts were analyzed by gel electrophoresis. For fractionation after import, intact chloroplasts were washed 2 times with import buffer, then diluted with HL buffer (10 mM HEPES-KOH, 10 mM MgCl₂, pH=8) and total mixture was frozen in liquid nitrogen/thawed 3 times, then centrifuged at 16,000 g for 20 min. Supernatant, containing soluble chloroplast proteins, and the pellet, containing membrane fraction, were analyzed by SDS-PAGE gel. Alkaline treatment of membrane fraction was performed with 200 mM Na₂CO₃ for 10 min on ice. Membranes were separated from supernatant by centrifugation at 16,000 g for 20 min and analyzed by SDS-PAGE. Radiolabeled protein was analyzed by phosphorimaging (Storm, Molecular Dynamics).

Protoplast Isolation and Transient Expression:

Isolation and transformation of maize protoplasts were performed according to classical protocols with modifications. Maize var. B73 plants were grown in the dark at 26° C. for 12 days (Avantis growth chamber (Conviron)). Middle parts of 2nd leaves of 20 plants were cut into razor thin sections and transferred to 500 ml Erlenmeyer flask containing 50 ml of Ca/Mannitol solution (10 mM CaCl2, 0.6M Mannitol, 20 mM MES pH 5.7) to which was added 1% cellulase (Trichoderma viride), 0.3% pectinase (Rhizopus sp.) (Sigma), 5 mM β-mercaptoethanol (Sigma), and 0.1% BSA (Sigma). A vacuum was applied for 5 min followed by shaking at 60 rpm at room temperature in the dark for 3 hours. The supernatant was filtered by 60 μm nylon mesh, and collected in a 50 ml Falcon centrifuge tube by centrifugation at 500 g and then washed three times with Ca/mannitol solution (0.6 M mannitol, 10 mM CaCl₂, 20 mM MES pH 5.6). Purified pUC35S-_{Zm}CruP-GFP plasmid DNA (10 μg in 25 μl ice-cold water) was added to 1×10⁶ protoplasts in 100 μl ice-cold 0.6 M mannitol containing 10 mM CaCl₂, and immediately diluted with 500 μl of PEG solution (40% polyethylene glycol 6000, 0.5 M mannitol, 0.1 M Ca(NO₃)₂. The protoplast suspension was gently mixed for 10 to 15 sec, diluted with 4.5 ml mannitol/MES solution (0.5 M mannitol, 15 mM MgCl₂, 0.1% MES pH 5.6) and kept at room temperature for 20 min. The protoplasts were centrifuged at 500 g for 5 min at room temperature, the supernatant removed and the pellet was re-suspended in 1 ml of Ca/mannitol solution and incubated for 12-16 hrs at 25° C. Transient expression of the _{Zm}CruP:GFP fusion protein was visualized with a DMI6000B inverted confocal microscope with TCS SP5 system (Leica Microsystems CMS, Germany) using a water immersion objective (63×). A 488 nm argon laser was used as the excitation wavelength of GFP and chlorophyll. The chloroplast autofluorescence was detected between 664 and 696 nm, and the GFP fluorescence was detected between 500 and 539 nm and always confirmed by recording the emission spectrum by wavelength scanning (lambda scan) between 500 and 600 nm with a 3 nm detection window. LAS AF software (Leica Microsystems CMS, Germany) was used for image acquisition. Images were obtained by combining several confocal Z-planes that had each been subjected to deconvolution.

TABLE 1
Primers

| | | SEQ |
|---|---|---|
| | | ID |
| Name | Sequence 5′-3′ | NO |
| 1857 | TGGTTCACGTAGTGGGCCATCG | 1 |
| 1871 | ATTCCAAAGTCCCTGAAGTTGTTAC | 2 |
| 2190 | ATATTCCACTCCTGTTCCCTCCCT | 3 |
| 2292 | TTGCGTCATAGATTCCTTTT | 4 |
| 2293 | ACTTGTCACCAGTCCGTTGC | 5 |
| 2487 | ACTCTAGATGCCTCCGCCT | 6 |
| 2617 | GAGCGACAACCCGAAGACC | 7 |
| 2618 | AATCCATATGGAATCCCTAGC | 8 |
| 2930 | GATCTAGATTATTCCCCTTGGCGGTAATC | 9 |
| 2938 | GAGGATCCTTCCCCTTGGCGGTAATC | 10 |
| 2958 | GTCTCTAGATTATTTTTCAAATTGAGGATGAGACCATT | 11 |
| | CCCCTTGGCGGTAATCTAAACCG | |
| 2960 | ACTCGAGATGCCTCCGCCTGTTCTTC | 12 |
| 2965 | GATTCTAGATGCCTCCGCCTGTTCTT | 13 |
| 2978 | GGCTCTGGTCCATTAGACCGCA | 14 |
| 2979 | GCAGCTGGCAACGGACTATTTCG | 15 |
| 2986 | TATCGGACATATGGGTCAGG | 16 |
| 2987 | CGGGATCCAAGCTTAGTCCTGTTC | 17 |

### INCORPORATION OF SEQUENCE LISTING

Incorporated herein by reference in its entirety is the Sequence Listing for the application. The Sequence Listing is disclosed on a computer-readable ASCII text file titled, “sequence_listing.txt”, created on Nov. 21, 2014. The sequence_listing.txt file is 3 kb in size.

We claim:

1. A method for selecting a plant that is cold and anoxia tolerant, the method comprising detecting the expression of CruP in said plant, wherein a plant that overexpresses CruP is selected as being cold and anoxia tolerant.

2. The method according to claim 1, wherein the plant is algae.

3. A method for providing cold tolerance to a plant, the method comprising introducing a gene that encodes CruP with higher than wild-type expression into said plant.

4. The method of claim 3, wherein the plant is algae.

5. A method for providing anoxia tolerance to a plant, the method comprising introducing a gene that encodes CruP with higher than wild type expression into said plant.

6. The method of claim 5, wherein the plant is algae.

7. A method for providing protection against reactive oxygen species in a plant, the method comprising introducing a gene that encodes CruP with higher than wild type expression in said plant.

8. The method of claim 7, wherein the plant is algae.


SEQUENCE LISTING (17 sequences)

SEQ ID NO: 1 - DNA, 22 bp, Zea Mays
tggttcacgtagtgggccatcg

SEQ ID NO: 2 - DNA, 25 bp, Zea Mays
attccaaagtccctgaagttgttac

SEQ ID NO: 3 - DNA, 24 bp, Zea Mays
atattccactcctgttccctccct

SEQ ID NO: 4 - DNA, 20 bp, Zea Mays
ttgcgtcatagattcctttt

SEQ ID NO: 5 - DNA, 20 bp, Zea Mays
acttgtcaccagtccgttgc

SEQ ID NO: 6 - DNA, 19 bp, Zea Mays
actctagatgcctccgcct

SEQ ID NO: 7 - DNA, 19 bp, Zea Mays
gagcgacaacccgaagacc

SEQ ID NO: 8 - DNA, 21 bp, Zea Mays
aatccatatggaatccctagc

SEQ ID NO: 9 - DNA, 29 bp, Zea Mays
gatctagattattccccttggcggtaatc

SEQ ID NO: 10 - DNA, 26 bp, Zea Mays
gaggatccttccccttggcggtaatc

SEQ ID NO: 11 - DNA, 61 bp, Zea Mays
gtctctagattatttttcaaattgaggatgagaccattccccttggcggtaatctaaaccg

SEQ ID NO: 12 - DNA, 26 bp, Zea Mays
actcgagatgcctccgcctgttcttc

SEQ ID NO: 13 - DNA, 26 bp, Zea Mays
gattctagatgcctccgcctgttctt

SEQ ID NO: 14 - DNA, 22 bp, Zea Mays
ggctctggtccattagaccgca

SEQ ID NO: 15 - DNA, 23 bp, Zea Mays
gcagctggcaacggactatttcg

SEQ ID NO: 16 - DNA, 20 bp, Zea Mays
tatcggacatatgggtcagg

SEQ ID NO: 17 - DNA, 24 bp, Zea Mays
cgggatccaagcttagtcctgttc